(19) United States
(12) Patent Application Publication
(10) Pub. No.: US 2010/0041745 A1
(43) Pub. Date: Feb. 18, 2010

(54) Aigialomycin D and Derivatives Thereof and Their Use in Treating Cancer or Malaria or a Microbial Infection

(76) Inventors: Anqi Chen, Jurong Island (SG); Quang Vu Nguyen, Jurong Island (SG)

Correspondence Address: FENWICK & WEST LLP, SILICON VALLEY CENTER, 801 CALIFORNIA STREET, MOUNTAIN VIEW, CA, 94041, US

(21) Appl. No.: 12/374,610

(22) PCT Filed: Jul. 20, 2007
(86) PCT No.: PCT/SG2007/000216

§ 371 Date: Sep. 21, 2009

Related U.S. Application Data
(60) Provisional application No. 60/829,005, filed on Oct. 11, 2006.

(51) Int. Cl. A61K 31/335; C07D 313/06; A61P 35/00; A61P 33/06; A61P 31/00

(52) U.S. Cl. 514/450; 549/268

(57) ABSTRACT

The invention describes a process for making compound (2), comprising the step of cyclising diene (3). Compound (2) may be aigialomycin D or a derivative thereof or may be elaborated to make aigialomycin D or derivative thereof. Furthermore compound (2) or derivative thereof can be used in treating cancer or malaria or a microbial infection.

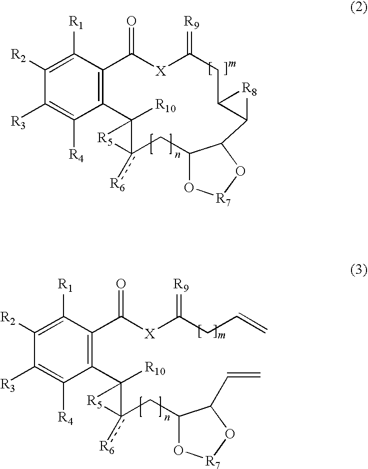

### TECHNICAL FIELD

The present invention relates to Aigialomycin D, derivatives thereof and synthesis of Aigialomycin D and derivatives thereof.

### BACKGROUND OF THE INVENTION

Aigialomycin D (1) is a 14-membered resorcinylic macrolides isolated from the marine mangrove fungus Aigialus parvus BCC5311.

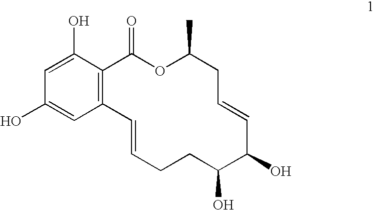

The structure of Aigialomycin D was elucidated by conventional structural determination methods and single crystal X-ray diffraction analysis after derivatisation. In terms of biological activity, aigialomycin D shows not only potent antitumour activity (IC₅₀: 1.8 μg/ml and 3.0 μg/ml against Vero and KB cells respectively) but also anti-malarial activity (IC₅₀: 6.6 μg/ml against P. falciparum). The biological target of aigialomycin D has just recently been identified as an inhibitor of kinases, in particular CDK and GSK-3 kinases. These biological properties of aigialomycin D coupled with its structural features have made this compound an attractive target for both synthetic studies and medicinal chemical exploration.

To date, there are three reported syntheses of aigialomycin D. Two of these are lengthy with low overall yields. A third synthesis disclosed a solid-phase synthesis strategy for aigialomycin D and analogues modified at the C5′-C6′ region whose inhibition on certain kinases was found to be less potent than aigialomycin D itself. The synthesis is difficult to scale up, and is capable of generating only limited analogues of aigialomycin D.

In view of the promising biological activities of aigialomycin D and the potential to discover more potent lead compounds from its parent structure, there remains a need to develop a more efficient and practical total synthetic route to aigialomycin D, its derivatives and analogues, which are crucial for the understanding of the mechanism of biological action and structure activity relationship (SAR) studies.

### OBJECT OF THE INVENTION

It is an object of the present invention to substantially overcome or at least ameliorate one or more of the above disadvantages. It is another object to at least partially satisfy the above need.

### SUMMARY OF THE INVENTION

In a first aspect of the invention there is provided a process for making compound 2:

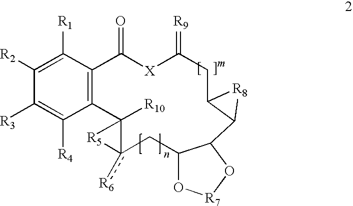

comprising the step of cyclising diene 3A:

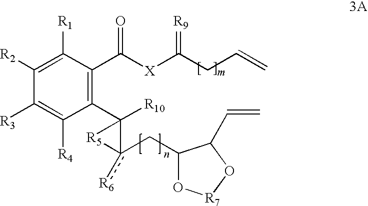

wherein:
m is an integer from 1 to 4; n is 0 or an integer from 1 to 4;
X is O or NRₐ wherein Rₐ is hydrogen, a protecting group, phenyl or an alkyl group of less than seven carbon atoms
R₁ and R₃ are, independently, OR_{b}, OC(O)R_{b} or OCO₂R_{b}, wherein each R_{b} is independently hydrogen, a protecting group, optionally substituted phenyl or an alkyl group of less than seven carbon atoms;
R₂ and R₄ are, independently, hydrogen, halogen, nitro, cyano, SR_{c}, N(R_{c})₂ or NC(O)R_{c}, wherein each Rc is, independently, hydrogen, a protecting group, optionally substituted phenyl or an alkyl group of less than seven carbon atoms; [symbol]
represents either a single bond or a double bond or a triple bond, whereby if it is a triple bond, R₅, R₆ and R₁₀ are all absent, and whereby, if it is a double bond, R₅ is absent, and, if it is a single bond, R₅ is present;
whereby, if [symbol] is a single bond, R₅ is a single bond, O, CH₂, CF₂, NR_{d} or NC(O)R_{d} wherein R_{d} is hydrogen, phenyl or an alkyl group of less than seven carbon atoms and R₆ is a hydrogen, alkyl (e.g. C1 to C6 alkyl), aryl (e.g. C6 to C14 aryl) or heteroaryl;
whereby, if R₅ is absent, R₆ is O, CH₂, CF₂, (H, F), (F, F), N—ORₑ, (H, ORₑ) or (OH, R_{f}) wherein Rₑ is hydrogen, alkyl sulfonyl, aryl sulfonyl or a protecting group, R_{f} is aryl, heteroaryl, alkyl or a perfluoroalkyl moiety of less than five carbon atoms;
R₇ is C═O, S═O, or a protecting group, or (H, H), or CRR′, wherein R and R′ are, independently, hydrogen, or an aryl group, or an alkyl or a cycloalkyl group, each of less than seven carbon atoms;
R₈ is a single bond

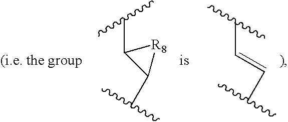

O, CH₂, CF₂, NRₕ or NC(O)Rₕ wherein Rₕ is hydrogen, phenyl or an alkyl group of less than seven carbon atoms; and R₉ is (H, R_{w}), where R_{w} is hydrogen, an alkyl group of less than 7 carbon atoms, an aryl group or a heteroaryl group;
R₁₀ is hydrogen or an alkyl or aryl group;
and wherein, where there is chirality at a position in compound 2 or diene 3A, the position may be in either R or S configuration or a mixture of both R and S configurations.

The following options may be used in the first aspect either individually or in any appropriate combination.

Diene 3A may be diene 3:

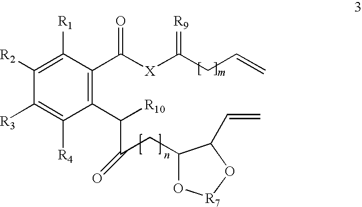

The group

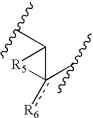

of diene 3A may be a CH₂C(═O) group.

The step of cyclising may be catalysed by a Grubbs catalyst or some other olefin metathesis catalyst, for example a carbene complex, particularly a transition metal carbene complex, or a ruthenium complex. The catalyst may be an N-heterocyclic carbene complex.

The process may comprise the step of making diene 3A by coupling alkene I and amide II:

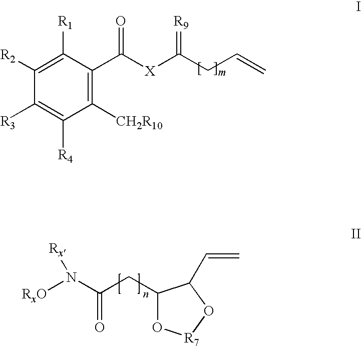

Rₓ and R_{x′} may each, independently, be an aryl group or an alkyl group of 1 to 6 carbon atoms. The step of coupling alkene I and amide II may comprise lithiation of the benzylic methylene group of alkene I (which is ortho to the C(═O)X group).

The process may comprise the step of making alkene I by coupling benzoic acid 4 with alkene 5:

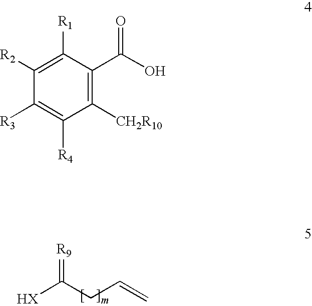

The step of coupling benzoic acid 4 with alkene 5 may comprise a Mitsunobu reaction or some other amide or ester forming reaction, for example a DCC coupling reaction, a transesterification reaction, an acid catalysed esterification, formation of an acid chloride followed by coupling with alkene 5 etc.

The process may additionally comprise elaboration of one or more functional groups of compound 2 so as to make aigialomycin D or a derivative thereof. The derivative may be:

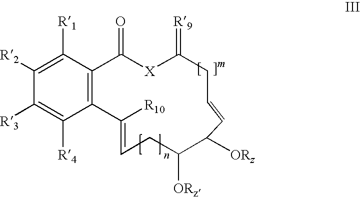

wherein:
R′₁, R′₂, R′₃, R′₄ and R′₉ are defined as for R₁, R₂, R₃, R₄ and R₉ respectively, and R_{z}, and R_{z′} are, independently, hydrogen, a protecting group, phenyl or an alkyl group of less than seven carbon atoms or R_{z} and R_{z′} together form a protecting group for a vicinal diol. The elaboration may comprise deprotection of one or more functional groups. The elaboration may comprise generation of a double bond in the non-aromatic ring.

In an embodiment there is provided a process for making compound 2 as defined above, said process comprising the step of cyclising diene 3, said cyclising being catalysed by a Grubbs catalyst.

In another embodiment there is provided a process for making compound 2 as defined above, said process comprising:

coupling alkene I and amide II to form diene 3; and

cyclising diene 3.

In another embodiment there is provided a process for making compound 2 as defined above, said process comprising:

coupling benzoic acid 4 with alkene 5 to make alkene I;

coupling alkene I and amide II to form diene 3; and

cyclising diene 3.

In another embodiment there is provided a process for making aigialomycin D or a derivative thereof, said process comprising:
coupling benzoic acid 4 with alkene 5 to make alkene I;
coupling alkene I and amide II to form diene 3;
cyclising diene 3 to make compound 2; and
elaborating of one or more functional groups of compound 2 so as to make aigialomycin D or the derivative thereof.

In another embodiment there is provided a process for making aigialomycin D or a derivative thereof, said process comprising:
coupling benzoic acid 4 with alkene 5 to make alkene I by means of a Mitsunobu reaction;
coupling alkene I and amide II to form diene 3 by a reaction comprising the step of lithiation of the benzylic methylene group of alkene I;
cyclising diene 3 to make compound 2, said cyclising being catalysed by a Grubbs catalyst; and
elaborating of one or more functional groups of compound 2 so as to make aigialomycin D or the derivative thereof.

The invention also provides aigialomycin D or a derivative thereof when made by the process of the first aspect.

In a second aspect of the invention there is provided the use of aigialomycin D or a derivative thereof when made by the process of the first aspect, in treating cancer or malaria or a microbial infection.

In a third aspect of the invention there is provided the use of aigialomycin D or a derivative thereof when made by the process of the first aspect, for the preparation of a medicament for the treatment of cancer or malaria or a microbial infection.

In a fourth aspect of the invention there is provided a method of treating cancer or malaria or a microbial infection, said method comprising administering to a patient in need thereof a therapeutically effective dose of aigialomycin D or a derivative thereof made by the process of the first aspect.

In a fifth aspect of the invention there is provided a process for making a medicament for the treatment of cancer or malaria or a microbial infection comprising:
making aigialomycin D or a derivative thereof by the process of the first aspect; and
combining said aigialomycin D or derivative thereof with one or more clinically or pharmaceutically acceptable carrier, diluent and/or adjuvant.

In a sixth aspect of the invention there is provided a method of treating cancer or malaria or a microbial infection, said method comprising making a medicament by the process of the fifth aspect and administering a therapeutically effective dose of said medicament to a patient in need thereof.

### DETAILED DESCRIPTION OF THE INVENTION

The present invention provides an efficient and practical route for the synthesis of aigialomycin D and related compounds using readily available starting materials. This route enables the preparation of aigialomycin D and a library of its designed analogues and derivatives, enabling the screening of these compounds for biological activity. These compounds are thought to have application as potential drugs for treatment of cancers and malaria.

In the present specification, the term “derivative” may refer to a compound actually derived from another, or may refer to a compound which is derivable therefrom, or to one which is structurally related thereto. In the present specification, the term “protecting group” refers to any one of those commonly used moieties for the protection of functional groups related to this invention. Examples are as those listed, but not limited to, in “T. W. Greene et al, Protective Groups in Organic Synthesis”, 3^{rd} Ed., 1999, John Wiley & Sons, Inc. the contents of which are incorporated herein by cross-reference. Some suitable protecting groups include:

in R₆═OC(O)Rₚ or OCO₂Rₚ, wherein Rₚ is hydrogen, allyl or optionally substituted phenyl or an alkyl group of less than seven carbon atoms;
in R₇, R_{z}, R_{z′} and Rₑ═SiRₜR_{t′}R_{tt″}, wherein Rₜ, R_{t′} and R_{tt″} are the same or independently Me, Et, Ph or tBu; or ═CORᵤ wherein Rᵤ is hydrogen, allyl or optionally substituted phenyl or an alkyl group of less than five carbon atoms; or =benzyl or substituted benzyl; or ═CH₂XR wherein X═O, S, R=Me, Et; or =tetrahydropyran (THP).

In the present specification, unless otherwise specified:

alkyl groups may be C1 to C6 straight chain, or C3 to C6 branched chain or cycloalkyl. Suitable examples include methyl, ethyl, propyl, isopropyl, butyl, cyclopropyl, cyclobutyl, cyclohexyl, cyclopropylmethyl etc.
aryl groups may be monocyclic, bicyclic, tricyclic etc. and may be fused or linked cyclic structures. Suitable groups include phenyl, naphthyl, anthracyl, biphenyl, acenaphthyl, terphenyl etc. They may optionally be substituted, e.g. with one or more alkyl or aryl groups.
heterocyclic groups may be monocyclic, bicyclic etc. and may be fused or linked cyclic structures, and may also be aromatic or non-aromatic, for example pyridyl, furyl, thiofuryl, oxazolinyl, pyrrolyl, quinolinyl, indolyl, pyrrolidinyl, piperazinyl etc.

Thus a versatile route to compounds of general structure 2 is provided.

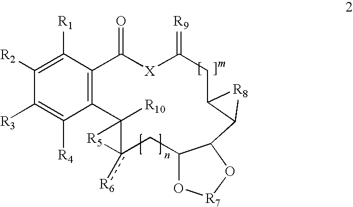

The compound of structure 2 may be a resorcinyl macrolide. It may be a macrocycle. It may comprise a macrocycle fused with an aromatic ring. It may comprise a non-aromatic ring fused with an aromatic ring. The non-aromatic ring may comprise at least about 14 atoms, or between about 12 and about 30 atoms or about 12 to 20, 12 to 18, 12 to 16, 12 to 14, 14 to 20, 14 to 18, 14 to 16, 16 to 30 or 20 to 30 atoms, e.g. about 12, 13, 14, 15, 16, 17, 18, 19, 20, 21, 22, 23, 24, 25, 26, 27, 28, 29 or 30 atoms. At least one of said atoms may be a heteroatom (i.e. not carbon). The compound of structure 2 may be an antitumour and/or anticancer agent. It may be an antimalarial. It may be an antibiotic. It may be capable of targeting an inhibitor of kinases, in particular CDK and GSK-3 kinases.

In structure 2, m may be 0 or an integer from 1 to 4, or 1 to 3, and may be 0, 1, 2, 3 or 4. n may be 0 or an integer from 1 to 4, or to 3, and may be 0, 1, 2, 3 or 4.

X may be O or NRₐ, so that the functional group in compound 2 incorporating X is an ester or an amide respectively. Rₐ may be hydrogen, a protecting group, phenyl (optionally substituted, for example with one or more alkyl and/or aryl groups) or an alkyl group of less than seven carbon atoms (e.g. 1, 2, 3, 4, 5 or 6 carbon atoms). The alkyl group may be straight chain, branched or cyclic. It may for example be methyl, ethyl, propyl, isopropyl, t-butyl, cyclopentyl, cyclopentylmethyl, benzyl, p-methylphenyl or some other suitable group.

[symbol] represents either a single bond or a double bond or a triple bond. If it is a triple bond, R₅ and R₆ are absent in all structures and R₁₀ is absent in structures 2 and 3A and is H in other structures if present. If it is a double bond, R₅ is absent, so that the group incorporating [symbol] is:

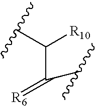

wherein R₆ is O, CH₂, CF₂, (H, F), (F, F), N—ORₑ, (H, ORₑ), (OH, R_{f}), CHR_{f}, (H, H), (H, R_{f}), (R_{f}, R_{f′}) or —(CH₂)ₙ. In the foregoing, the notation (A, B), (which is used throughout this specification) except where specifically indicated to the contrary, indicates that both A and B are bonded to the carbon atom by single bonds. Thus for example (H, F) would signify that the group is:

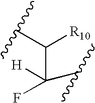

Rₑ in R₆ may be hydrogen, alkyl sulfonyl, aryl sulfonyl or a protecting group. R_{f} and R_{f′} may, independently, be aryl, heteroaryl, alkyl or a perfluoroalkyl moiety of less than five carbon atoms, e.g. 1, 2, 3 or 4 carbon atoms. The alkyl groups (of either Rₑ or R_{f} or R_{f′}) may, independently, be C1 to C12 straight chain alkyl groups, or C3 to C12 branched chain or cyclic alkyl groups. The aryl groups may be optionally substituted phenyl, biphenyl, terphenyl, fused aryl (e.g. naphthyl, phenanthryl, acenaphthyl) or other aryl group. The heteroaryl group may be for example pyridyl, oxazolinyl, isoxazolinyl, furyl, thiophenyl etc.

If [symbol] is a single bond, R₅ is present, so that the group incorporating [symbol] is:

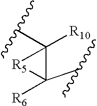

In this case R₅ may be O, CH₂, CHR_{d}, CF₂, NR_{d} or NC(O)R_{d} wherein R_{d} is hydrogen, a protecting group, phenyl (optionally substituted, for example with one or more alkyl and/or aryl groups) or an alkyl group of less than seven carbon atoms (e.g. 1, 2, 3, 4, 5 or 6 carbon atoms). The alkyl group may be straight chain, branched or cyclic. It may for example be methyl, ethyl, propyl, isopropyl, t-butyl, cyclopentyl, cyclopentylmethyl, benzyl, p-methylphenyl or some other suitable group. R₆ in this case is a hydrogen, alkyl (e.g. C1 to C6 alkyl), aryl (e.g. C6 to C14 aryl) or heteroaryl (e.g. pyridyl, furyl, thiofuryl, pyrrolyl etc.). R₅ may be a bond, forming part of a double bond, so that the group incorporating [symbol] may be:

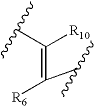

Whereas the trans form of the double bond is shown above, the double bond may be either in the cis form or in the trans form.
R₁ and R₃ may, independently, be OR_{b}, OC(O)R_{b} or OCO₂R_{b}. Each R_{b} may, independently, be hydrogen, a protecting group, phenyl (optionally substituted, for example with one or more alkyl and/or aryl groups) or an alkyl group of less than seven carbon atoms (e.g. 1, 2, 3, 4, 5 or 6 carbon atoms). The alkyl group may be straight chain, branched or cyclic. It may for example be methyl, ethyl, propyl, isopropyl, 1-butyl, cyclopentyl, cyclopentylmethyl, benzyl, p-methylphenyl or some other suitable group. Suitable protecting groups are described elsewhere in this specification.

R₂ and R₄ may, independently, be hydrogen, halogen, nitro (NO₂), cyano (CN), SR_{c}, OR_{c}, N(R_{c})₂, NR_{c}R_{c′} or NC(O)R_{c}, wherein each R_{c} and R_{c′} is, if present, independently, hydrogen, a protecting group, phenyl (optionally substituted, for example with one or more alkyl and/or aryl groups) or an alkyl group of less than seven carbon atoms (e.g. 1, 2, 3, 4, 5 or 6 carbon atoms). The alkyl group may be straight chain, branched or cyclic. It may for example be methyl, ethyl, propyl, isopropyl, t-butyl, cyclopentyl, cyclopentylmethyl, benzyl, p-methylphenyl or some other suitable group.

R₇ may be C═O, S═O, or a protecting group, or (H, H) or CRR′. R and R′ here may be, independently, hydrogen, aryl or alkyl or cycloalkyl. The alkyl group may be straight chain or branched or cyclic. A suitable branched chain alkyl or cycloalkyl group may have 3 to 6 carbon atoms, e.g. 3, 4, 5 or 6 carbon atoms, and a suitable straight chain alkyl group may have 1 to 6 carbon atoms, e.g. 1, 2, 3, 4, 5 or 6 carbon atoms. Aryl groups may include for example optionally substituted phenyl, naphthyl, anthracyl, biphenyl, terphenyl etc. The terminology (H, H) here indicates that each oxygen atom is attached to a hydrogen atom, i.e. the group incorporating R₇ is:

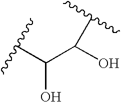

R₈ may be O, CH₂, CF₂, NRₕ or NC(O)Rₕ wherein Rₕ is hydrogen, phenyl (optionally substituted, for example with one or more alkyl and/or aryl groups) or an alkyl group of less than seven carbon atoms (e.g. 1, 2, 3, 4, 5 or 6 carbon atoms). The alkyl group may be straight chain, branched or cyclic. It may for example be methyl, ethyl, propyl, isopropyl, t-butyl, cyclopentyl, cyclopentylmethyl, benzyl, p-methylphenyl or some other suitable group. Alternatively R₈ may be a bond, so that a double bond is present and the group incorporating R₈ is:

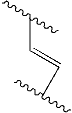

Whereas the trans form of the double bond is shown above, the double bond may be in either cis form or in the trans form.

R₉ is (H, R_{w}), where R_{w} is hydrogen or an alkyl group of less than 7 carbon atoms, and may have 1, 2, 3, 4, 5 or 6 carbon atoms (e.g. methyl, ethyl, propyl, butyl, isopropyl etc.) or an optionally substituted aryl or heteroaryl group i.e. the group incorporating R₉ may be:

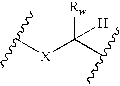

R₁₀ is hydrogen or an alkyl or aryl group. The alkyl group may be C1 to C6 straight chain (e.g. methyl, ethyl, propyl, butyl, pentyl or hexyl), or C3 to C6 branched chain or cycloalkyl (e.g. isopropyl, isobutyl, t-butyl, cyclopropyl, cyclohexyl, cyclopentylmethyl). The aryl group may be monocyclic, bicyclic, tricyclic etc. and may be fused or linked. Suitable groups include phenyl, naphthyl, anthracyl, biphenyl, acenaphthyl, terphenyl etc. They may optionally be substituted, e.g. with one or more alkyl or aryl groups.

In the above structures, and other structures described herein, where there is a chiral centre (e.g. an asymmetric carbon atom), that chiral centre may be in either R or S configuration or a mixture of both R and S configurations. Thus a single structure as described above may describe a variety of diastereomeric compounds and/or optical isomers, and also incorporates mixtures of any two or more of these.

Some examples of various options for the different groups in the structures of the invention are shown in the table below, although it should be recognised that these are examples only and are intended to illustrate the present invention. The table is not intended to be an exhaustive list of options. Many options as defined earlier are also envisaged in the present invention.

| R₁ | R₂ | R₃ | R₄ | [symbol] | R₅ | R₆ | R₇ | R₈ | R₉ | R₁₀ | X | m | n |
|---|---|---|---|---|---|---|---|---|---|---|---|---|---|
| MOMO | H | MOMO | H | — | — | H | CMe₂ | — | (H, Me) | H | O | 1 | 2 |
| MOMO | H | MOMO | H | ═ | none | O | CMe₂ | — | (H, Me) | H | O | 1 | 2 |
| MOMO | H | MOMO | H | ═ | none | NMe | CMe₂ | — | (H, Me) | H | O | 1 | 2 |
| MOMO | H | MOMO | H | — | — | H | CMe₂ | — | (H, Me) | H | NMe | 1 | 2 |
| MOMO | H | MOMO | H | ═ | none | O | CMe₂ | — | (H, Me) | H | NMe | 1 | 2 |
| OH | H | OH | H | — | — | H | CMe₂ | — | (H, Me) | H | O | 1 | 2 |
| OH | H | OH | H | ═ | none | O | CMe₂ | — | (H, Me) | H | O | 1 | 2 |
| MOMO | Cl | MOMO | H | — | — | H | CMe₂ | — | (H, Me) | H | O | 1 | 2 |
| MOMO | Cl | MOMO | H | ═ | none | O | CMe₂ | — | (H, Me) | H | O | 1 | 2 |
| MOMO | H | MOMO | Br | — | — | H | CMe₂ | — | (H, Me) | H | O | 1 | 2 |
| MOMO | H | MOMO | Br | ═ | none | O | CMe₂ | — | (H, Me) | H | O | 1 | 2 |
| MOMO | H | MOMO | H | — | — | H | CMe₂ | — | (H, Me) | H | O | 2 | 2 |
| MOMO | H | MOMO | H | ═ | none | O | CMe₂ | — | (H, Me) | H | O | 2 | 2 |
| MOMO | H | MOMO | H | — | — | H | CMe₂ | — | (H, Me) | H | O | 1 | 3 |
| MOMO | H | MOMO | H | ═ | none | O | CMe₂ | — | (H, Me) | H | O | 1 | 3 |
| MOMO | H | MOMO | H | — | — | H | CHMe | — | (H, Me) | H | O | 1 | 2 |
| MOMO | H | MOMO | H | ═ | none | O | CHMe | — | (H, Me) | H | O | 1 | 2 |
| MOMO | H | MOMO | H | — | — | H | CMe₂ | O | (H, Me) | H | O | 1 | 2 |
| MOMO | H | MOMO | H | ═ | none | O | CMe₂ | O | (H, Me) | H | O | 1 | 2 |
| MOMO | H | MOMO | H | — | — | H | CMe₂ | — | (H, Ph) | H | O | 1 | 2 |
| MOMO | H | MOMO | H | ═ | none | O | CMe₂ | — | (H, Ph) | H | O | 1 | 2 |
| MOMO | H | MOMO | H | — | CH₂ | H | CMe₂ | — | (H, Me) | H | O | 1 | 2 |
| MOMO | H | MOMO | H | — | O | H | CMe₂ | — | (H, Me) | H | O | 1 | 2 |
| MOMO | H | MOMO | H | — | — | H | CMe₂ | — | (H, Me) | Me | O | 1 | 2 |
| MOMO | H | MOMO | H | ═ | none | O | CMe₂ | — | (H, Me) | Ph | O | 1 | 2 |
| MOMO | H | MOMO | H | — | — | H | CMe₂ | — | (H, H) | H | O | 1 | 2 |
| MOMO | H | MOMO | H | ═ | none | O | CMe₂ | — | (H, H) | H | O | 1 | 2 |

In the synthesis described herein, there may be one or more steps of protection and/or deprotection of functional groups. These steps of protection and deprotection are well known in the art. It will be readily understood that the steps of protection and deprotection may occur at different stages through the synthesis, depending on the requirements of the various functional groups for protection, and the various appropriate options for timing of protection and/or deprotection steps are encompassed by the present invention.

Some common protecting groups are set out below. These and/or other protecting groups may be used as appropriate in the present invention.

### Protecting Groups for OH Groups:

acetyl—these may be removed by either acid or base
alkoxyethers or thioethers: e.g. β-methoxyethoxymethyl ether (MEM), methoxymethyl ether (MOM), methyl thiomethyl ether—these may be removed by acid
p-methoxybenzyl ether (PMB)—these may be removed by acid, hydrogenolysis, or oxidation
pivaloyl—these may be removed by acid, base or reductant agents
tetrahydropyran (TBP)—these may be removed by acid
silyl ethers e.g. trimethylsilyl (TMS), tert-butyldimethylsilyl (TBDMS), triisopropylsilyl (TIPS) ethers—these may be removed by acid or fluoride ion, e.g. e.g. sodium fluoride or tetraalkylammonium fluoride
methyl ethers—these may be removed using trimethylsilyl iodide or boron tribromide

### Protecting Groups for Amines:

carbobenzyloxy (Cbz) group—these may be removed by hydrogenolysis
tert-butyloxycarbonyl (BOC) group—these may be removed by concentrated, strong acid
9-fluorenylmethyloxycarbonyl (FMOC)—these may be removed by base
benzyl (Bn) group—these may be removed by hydrogenolysis
p-methoxyphenyl (PMP) group—these may be removed by ammonium cerium (IV) nitrate (CAN)

### Protecting Groups for Carboxylic Acid

methyl esters—these may be removed by acid or base
benzyl esters—these may be removed by hydrogenolysis
tert-butyl esters—Removed by acid, base and some reductants
silyl esters—these may be removed by acid, base and organometallic reagents

### Protecting Groups for Vicinal Diols

geminal diethers (acetals or ketals) e.g. acetonyl (2,2-propanediyl ethers)—these may be removed by acid
benzaldehyde acetals—these may be removed by hydrogenolysis.

The key step of the synthesis of compound 2 is the cyclisation of diene 3A to form compound 2. The cyclisation comprises forming a double bond between a carbon atom of each of the terminal double bonds of 3A (e.g. between the non-terminal carbon atoms thereof) so as to complete a ring. The cyclisation may be catalysed by a catalyst. The catalyst may be a carbene complex. The carbene complex may be a transition metal carbene complex. It may be a ruthenium complex. It may be au N-heterocyclic carbene complex. The catalyst may be a Grubbs catalyst. It may be a 1^{st} or 2^{nd} generation Grubbs catalyst. It may be a Hoveyda-Grubbs catalyst. The catalyst may be for example benzylidene-bis(tricyclohexylphosphine)dichlororuthenium or benzylidene[1,3-bis(2,4,6-tethylphenyl)-2-idazolidinylidene]dichloro(tricyclohexylphosphine)ruthenium. The cyclisation may be a ring-closing metathesis reaction. The reaction may use a concentration of catalyst between about 5 and about 30 mol % relative to diene 3A, or about 5 to 20, 5 to 10, 10 to 30, 10 to 20 or 20 to 30%, e.g. about 5, 10, 15, 20, 25 or 30%. It may be conducted at about 30 to about 120° C., or about 30 to 100, 30 to 50, 50 to 120, 100 to 120, 40 to 100, 40 to 80 or 80 to 120° C., e.g. about 30, 35, 40, 45, 50, 55, 60, 65, 70, 75, 80, 85, 90, 95, 100, 105, 110, 115 or 120° C. Depending on solvent, it may be necessary to increase the pressure to attain such temperatures. The pressure may be between about 1 and about 10 atm, or about 1 to 5, 1 to 2, 2 to 10, 5 to 10 or 2 to 6 atm, e.g. about 1, 2, 3, 4, 5, 6, 7, 8, 9 or 10 atm. The reaction may take between about 0.2 and about 60 hours, depending on temperature, solvent, catalyst and substrate, and may take about 0.2 to 30, 0.2 to 20, 0.2 to 10, 0.2 to 1, 0.2 to 0.5, 0.5 to 60, 1 to 60, 10 to 60, 20 to 60, 30 to 60, 1 to 50, 1 to 20, 1 to 10, 10 to 50 or 30 to 50 hours, e.g. about 0.2, 0.3, 0.4, 0.5, 0.6, 0.7, 0.8, 0.9, 1, 2, 3, 4, 5, 6, 7, 8, 9, 10, 11, 12, 18, 24, 30, 36, 42, 48, 54 or 60 hours. The reaction may provide a yield under appropriate conditions of at least about 75%, or at least about 80, 85, 90, 95 or 98%. The reaction may generate a mixture of double bond isomers having at least 50% E, or at least about 60, 70, 80, 90, 95 or 99% E, e.g. about 50, 60, 70, 80, 85, 90, 95, 96, 97, 98 or 99%, or may generate 100% E isomer. Suitable solvents include polar aprotic solvents such as diethyl ether, dichloromethane, dichloroethane, chloroform, etc. or mixtures thereof.

Diene 3 may be used in the cyclisation step described above, or may be used to make diene 3A for use in the cyclisation step. Conversion of the CH₂C(═O) group of diene 3 to the styrenic double bond of a diene 3A in which [symbol] is a double bond may be accomplished by reduction of the carbonyl group to a alcohol and dehydration of the resulting alcohol (optionally via an intermediate ester, ether or other suitable group). This sequence is well known in the literature. It may for example comprise formation of a mesityl ester and elimination of the ester to form the double bond.

Diene 3 may be made by coupling alkene I and amide II:

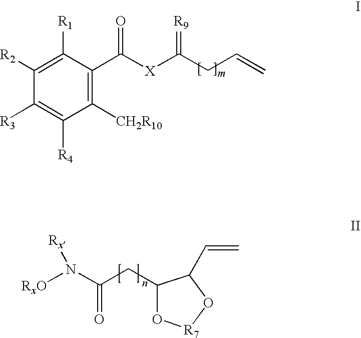

The nature of Rₓ and R_{x′} is in general not critical as they are not retained in diene 3. Commonly these will both be methyl groups, however ethyl, propyl, isopropyl, phenyl, benzyl etc. groups may also be used. Commonly they will be the same, however different groups are also contemplated by the present invention. The reaction of I and II to diene 3 commonly involves initially abstracting a benzylic hydrogen atom from I to form an anion. This requires use of a strong base, such as lithium diisopropylamide (LDA) although other strong bases may also be used. LDA may be generated in situ from diisopropylamine and n-butyl lithium. The reaction is commonly conducted at reduced temperature, e.g. between about −50 and about −100° C., or about −50 to −80, −70 to −100 or −70 to −90°, e.g. −50, −60, −70, −80, −90 or −100° C. A suitable temperature is about −78° C. Suitable solvents include common aprotic solvents, such as tetrahydrofuran. The solvent should be liquid at the temperature of the reaction. Following abstraction of the benzylic hydrogen atom to form an anion of I, II may be added, commonly without warming the reaction mixture appreciably and commonly without isolating any intermediate products, resulting in formation of diene 3.

Alkene I, for use in the above sequence, may be made by reaction of the corresponding benzoic acid 4 with olefin 5.

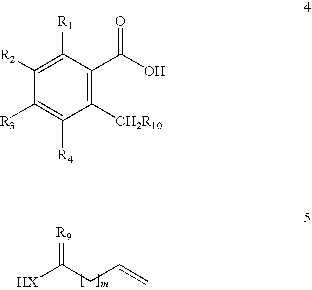

In some embodiments, X of compound 5 is O and R₉ is (H, CH₃), so that the olefin is a terminally unsaturated alcohol. In some embodiments R₂ and R₄ are H and R₁ and R₃ are MOMO groups (methoxymethoxy), which serve as protecting groups for phenolic OH groups. In the latter case, benzoic acid 4 may be made by first esterifying the carboxyl group, then forming the MOMO groups from the corresponding phenolic OH groups, then hydrolysing the ester group, so as to form the benzoic acid having protected phenolic groups on the ring. In some embodiments, R₁₀ is H, so that compound 4 is an orthotoluic acid derivative.

The coupling of 4 and 5 may be by means of the Mitsunobu reaction. Thus reaction of benzoic acid 4 with olefin 5, having an active hydrogen atom, may be conducted in the presence of a triarylphosphine (e.g. triphenylphosphine) and dialkyl (e.g. diethyl, diisopropyl or di-t-butyl) azodicarboxylate. Alternatives to this include use of heterogeneous (e.g. resin bound) triarylphosphine. A further alternative uses a phosphorane ylide NC—CH═PR₃ in place of the triaryl phosphine and dialkyl azodicarboxylate. Suitable ylides include (cyanomethylene)trimethylphosphorane and (cyanomethylene)tributylphosphorane. Suitable solvents for the coupling reaction include dipolar aprotic solvents, such as tetrahydrofuran, tetrahydropyran, ethylene carbonate, propylene carbonate etc. The reaction may be conducted at room temperature, or at any is suitable temperature, e.g. between about 10 and about 50° C. (or about 10 to 40, 10 to 30, to 50, 30 to 50 or 20 to 40° C., e.g. about 10, 15, 20, 25, 30, 35, 40, 45 or 50° C.). The reaction may be conducted in an inert atmosphere, e.g. under nitrogen, argon, etc. It may take between about 0.5 and about 5 days, or about 0.5 to 2, 0.5 to 1, 1 to 5, 2 to 5 or 1 to 3 days, e.g. about 0.5, 1, 1.5, 2, 2.5, 3, 3.5, 4, 4.5 or 5 days.

Other alternatives for the coupling of 4 and 5 are well known in the literature, For example, conversion of benzoic acid 4 to the corresponding benzoyl chloride activates it to reaction with compound 5, either with X being O or X being NRₐ. Other alternatives for this reaction include DCC mediated coupling of an acid to an alcohol. Also acid 4 may be converted to an anhydride (either symmetrical or a mixed anhydride, for example with a simple acid such as acetic acid) and reaction of the anhydride with olefin 5.

As noted above, in the event that one or more of R₁ to R₄ contain active hydrogen groups, these may be protected by protecting groups prior to conducting the coupling reaction. For example, if any of these are OH, they may be protected as methoxymethoxy groups (OCH₂OCH₃). The protecting group may be retained through one or more subsequent steps of the synthesis, and may be deprotected if and when desired (e.g. in dilute acid if methoxymethoxy groups are used).

Amide II may be made by standard organic chemical processes from a lactone

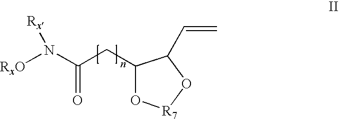

having a protected vicinal diol. A suitable protecting group is an acetonyl group as described above although other protecting groups, as described herein, may also be suitable. Reduction of the carbonyl group of the lactone provides a cyclic ether having an a-hydroxy group and a protected diol (i.e. a lactol having a protected diol). This reduction may be conveniently conducted using DIBAL-H (diisobutylaluminum hydride) usually at reduced temperature, e.g. about −50 to about −100° C., commonly at about −78° C. Reaction of the lactol with a Wittig reagent bearing a carboxylate ester functionality (for example Ph₃P═CHCO₂Et) in the presence of an acid (e.g. benzoic acid) provides a ring-opened acrylic acid derivative, bearing a terminal OH group and retaining the protected diol. The double bond of the acrylic functionality may be reduced using hydrogen gas and a suitable catalyst (e.g. Pd/C). Mild oxidation of the alcohol functionality by known methods provides an aldehyde, which can be elaborated to a terminal olefin by use of a Wittig reaction using Ph₃P═CH₂ (which may be generated in situ from Ph₃P⁺—CH₃Br⁻ and strong base such as butyl lithium). The ester functionality can then be converted to the desired amide functionality by reaction with the appropriate hydroxylamine derivative R_{x′}NHORₓ.HCl. A suitable scheme for this series of reactions is shown in FIG. 3.

The process may additionally comprise elaboration of one or more functional groups of compound 2 so as to make aigialomycin D or a derivative thereof. In the present context, the term “elaboration” refers to modifying or changing the group to a similar or related group. This may comprise for example removing one or more protecting groups in the compound 2. Methods for removing common protecting groups have been described earlier. The process may also comprise elaborating a styrenic double bond to generate for example an epoxide ring (e.g. using metachloroperbenzoic acid), a cyclopropyl ring (e.g. by means of an in situ generated carbene), a difluorocyclopropyl ring (e.g. by means of an in situ generated difluorocarbene), an aziridine ring (e.g. by means of an in situ generated nitrene) or some other suitable group. It may comprise converting a carbonyl group to a methylene or difluoromethylene group, or replacing it by two substituents. Each of the two substituents may, independently, be for example fluorine, hydrogen, OH or an alkoxy group. Other elaborations may also be used, and more than one elaboration may be required to obtain a desired derivative. The transformations used to achieve these elaborations are well known in the organic chemistry literature.

A generalised synthesis according to the present invention is shown in FIG. 1A. In FIG. 1A, conditions a may be any suitable conditions for forming an ester or an amide. Suitable esterification conditions include use of a triaryl phosphine (or other similar compound) and dialkylazodicarboxylate in a polar solvent at about 10-50° C. under an inert atmosphere for about 0.5 to 5 days. Alternatively, the benzoic acid may be converted to an acid chloride by means of thionyl chloride, and the resulting acid chloride exposed in an aprotic solvent to olefin 5. Further alternatives include use of DCC (dicyclohexylcarbodiimide) to effect the coupling reaction. Conditions b involve initially exposing compound I to a strong base. This is commonly done at about −50 to about −100° C. in an ether solvent, e.g. THF. Suitable bases include LDA, although others may be used, depending on the nature of compound I, for example sodium t-butoxide, sodium hydride etc. Following formation of the anion of compound I, it is exposed to compound II, generally without isolating or warming the anion. Conditions c depend greatly on the nature of the conversion, and in some instances compound 3 may be converted directly to compound 2 (in which R₆ is ═O). Conversion of 3 to 3A may involve initial reaction with sodium borohydride or other mild reducing agent, e.g. in alcohol/water at room temperature to generate compound 3A where R₅ is (H, H) and R₆ is OH. This may be elaborated if required with known chemistry, e.g. by mesylation with MsCl and triethylamine, and subsequent treatment with DBU or other base at elevated temperature followed if necessary by mild acid conditions to produce an olefin. Alternatively the alcohol product may be esterified or etherified or otherwise elaborated. The olefin may be further elaborated e.g. by cyclopropanation (e.g. using methylene iodide and diethyl zinc or metal catalysis, or other known method), epoxidation (e.g. using metachloroperbenzoic acid). Conditions d for the cyclisation involve reaction with a suitable catalyst, e.g. Grubbs catalyst or an N-heterocyclic carbene catalyst (optionally a polymeric N-heterocyclic carbene catalyst), commonly at about 5 to 30 mol %, at about 30 to about 120° C. and 1 to 10 atmospheres pressure for about 0.2 to 60 hours. A suitable solvent for this reaction is dichloromethane or chloroform. Further elaboration of the cyclised product may also be conducted. The conditions will depend on the nature of the transformation. They may involve deprotection (see elsewhere in this specification) or functional group modification, for example as described above for conditions c.

The invention also provides aigialomycin D or a derivative thereof when made by the process described above. As noted earlier, the present invention is capable of providing a wide range of derivatives of aigialomycin D, which may be used for their specific biological activities. It will be understood that other analogues of aigialomycin D may be made using the methodology described above. Further modification at the aromatic region may be used, for example, by replacing a heteroaryl or a heterocycle for the aromatic ring, and replacement of the C8′-C9′ (styrenic) double bond with a heterocycle. In order to achieve these modifications, it would be necessary to start with the corresponding starting materials, the structures of which will be readily apparent from the desired target product.

Aigialomycin D or a derivative thereof, made as described above, may be used in treating cancer or malaria or a microbial infection. It is well known that minor structural modifications to active compounds may enhance or modify the biological activity of a compound, and it is expected that derivatives of aigialomycin D, made available in quantity by the present invention, have useful biological activities.

In particular aigialomycin D or a derivative thereof may be used for the preparation of a medicament for the treatment of cancer or malaria or a microbial infection. The preparation may comprise combining the aigialomycin D or derivative thereof with one or more clinically acceptable carrier, diluent and/or adjuvant. Such carriers, diluents and/or adjuvants are in general well known.

Aigialomycin D or a derivative thereof, made as described above may be administered as compositions either therapeutically or preventively. In a therapeutic application, compositions are administered to a patient already suffering from a disease, in an amount sufficient to cure or at least partially arrest the disease and its complications. The composition should provide a quantity of the compound or agent sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 11.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraspinal, subcutaneous or intramuscular), oral or topical route. More preferably administration is by the parenteral route.

The carriers, diluents and adjuvants must be, “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension or emulsion or microemulsion, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, as preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The invention also provides a method of treating cancer or malaria or a microbial infection, said method comprising administering to a patient in need thereof a therapeutically effective dose of aigialomycin D or a derivative thereof made by the process of the invention as described above. The patient may be a human patient or may be a non-human patient. The patient may be a vertebrate. The vertebrate may be a mammal, a marsupial or a reptile. The mammal may be a primate or non-human primate or other non-human mammal. The mammal may be selected from the group consisting of human, non-human primate, equine, murine, bovine, leporine, ovine, caprine, feline and canine. The mammal may be selected from a human, horse, cattle, cow, ox, buffalo, sheep, dog, cat, goat, llama, rabbit, ape, monkey and a camel, for example. The patient may be a domesticated animal. It may be a pet. It may be a farm animal.

### BRIEF DESCRIPTION OF THE DRAWINGS

A preferred embodiment of the present invention will now be described, by way of an example only, with reference to the accompanying drawings wherein:

FIG. 1 depicts the general synthetic strategy for compounds (2);

FIG. 1A depicts a generalised synthetic scheme for compounds (2);

FIG. 2 depicts the synthetic route for aigialomycin D (1);

FIG. 3 depicts the synthesis of the amide (9);

FIG. 4 depicts the synthesis of the macrocyclisation precursor (18);

FIG. 5 depicts the synthesis of aigialomycin D (1); and

FIG. 6 depicts the synthesis of selected compounds (2).

### DETAILED DESCRIPTION OF THE PREFERRED EMBODIMENTS

The preferred embodiments of the present invention provide a synthesis of aigialomycin D (1) and compounds having the general structure (2)

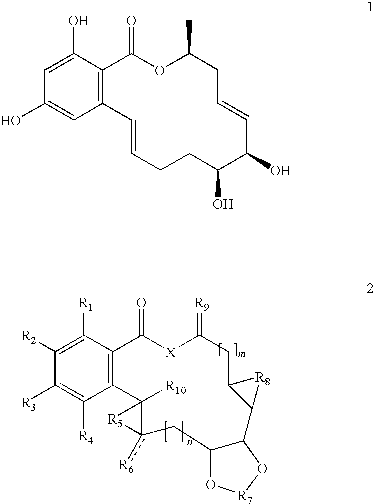

wherein:
m is an integer from 1-4; n is 0 or an integer from 1-4;
X is O, NRₐ wherein Rₐ is hydrogen, a protecting group, phenyl or an alkyl group of less than seven carbon atoms;
the dotted line represents a bond related to R₅, whereby, if it is a bond linked to R₆, R₅ is absent, and if it is absent, R₅ is present;
R₁ and R₃ are OR_{b}, OC(O)R_{b} or OCO₂R_{b}, wherein each R_{b} is independently hydrogen, a protecting group, phenyl or an alkyl group of less than seven carbon atoms;
R₂ and R₄ are independently hydrogen, halogen, nitro group, cyano group, SR_{c}, N(R_{c})₂ or NC(O)R_{c} wherein R_{c} is independently hydrogen, a protecting group, phenyl or an alkyl group of less than seven carbon atoms;
whereby, if the dotted line is absent, R₅ is a single bond, O, CH₂, CF₂, NR_{d} or NC(O)R_{d} wherein R_{d} is hydrogen, phenyl or an alkyl group of less than seven carbon atoms and R₆ is a hydrogen;
whereby, if R₅ is absent, R₆ is O, CH₂, CF₂, (H, F), (F, F), N—ORₑ, (H, ORₑ), (OH, R_{f}) wherein Rₑ is hydrogen, alkyl sulfonyl, aryl sulfonyl or a protecting group, R_{f} is aryl, heteroaryl, alkyl or a perfluoroalkyl moiety of less than five carbon atoms;
R₇ is C═O, S═O, or a protecting group;
R₈ is a single bond, O, CH₂, CF₂, NRₕ or NC(O)Rₕ wherein Rₕ is hydrogen, phenyl or an alkyl group of less than seven carbon atoms;

### R₉ is H, CH₃;

R₁₀ is hydrogen; and
wherein, where there is chirality at a position in the compound, it refers independently to both R and S configurations;

### General Synthetic Methodology

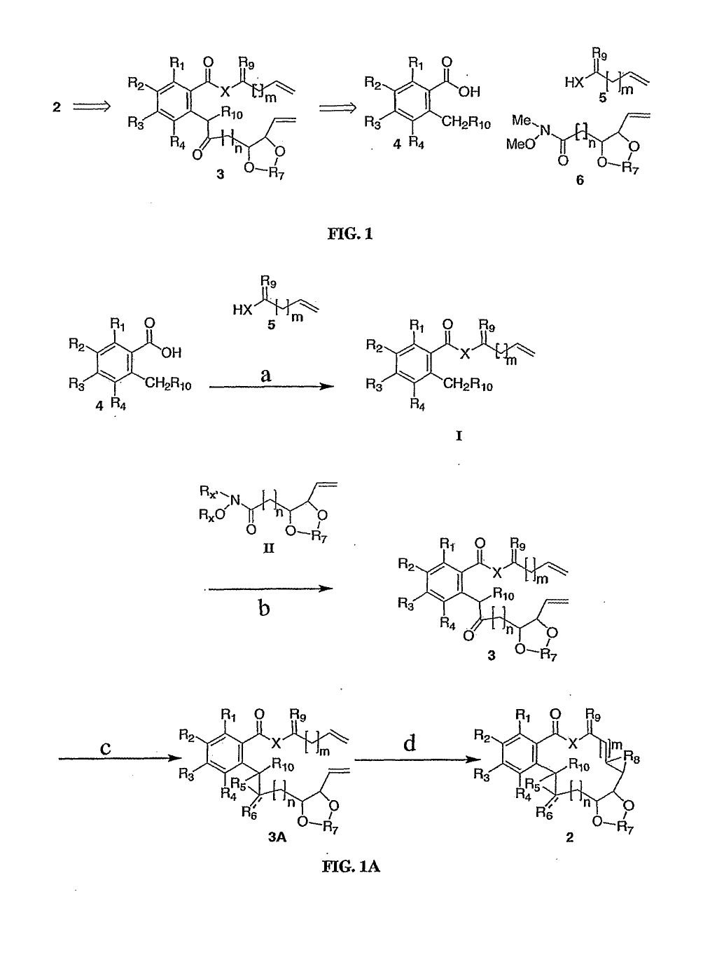

The general synthesis methodology for compound (2) is illustrated in FIG. 1. The cyclisation precursor (3) is prepared by coupling a substituted benzoic acid (4) and (5) by the Mitsunobu reaction, followed by acylation at the benzylic position in (4) with the amide (6) after lithiation at this position using LDA. The macrocyclisation is achieved on (3) by a ring closing metathesis (RCM) using Grubbs second generation catalyst to provide (2) (wherein R₅ is absent, the dotted line is a bond, R₆ is O and R₈ is a single bond). The installation of R₁-R₆ (except R₆═O) and R₈ is either carried out before assembly of (4)-(6) or after formation the key intermediate (2) (wherein R₅ is absent, the dotted line is a bond, R₆ is O and R₈ is a single bond).

### EXAMPLES

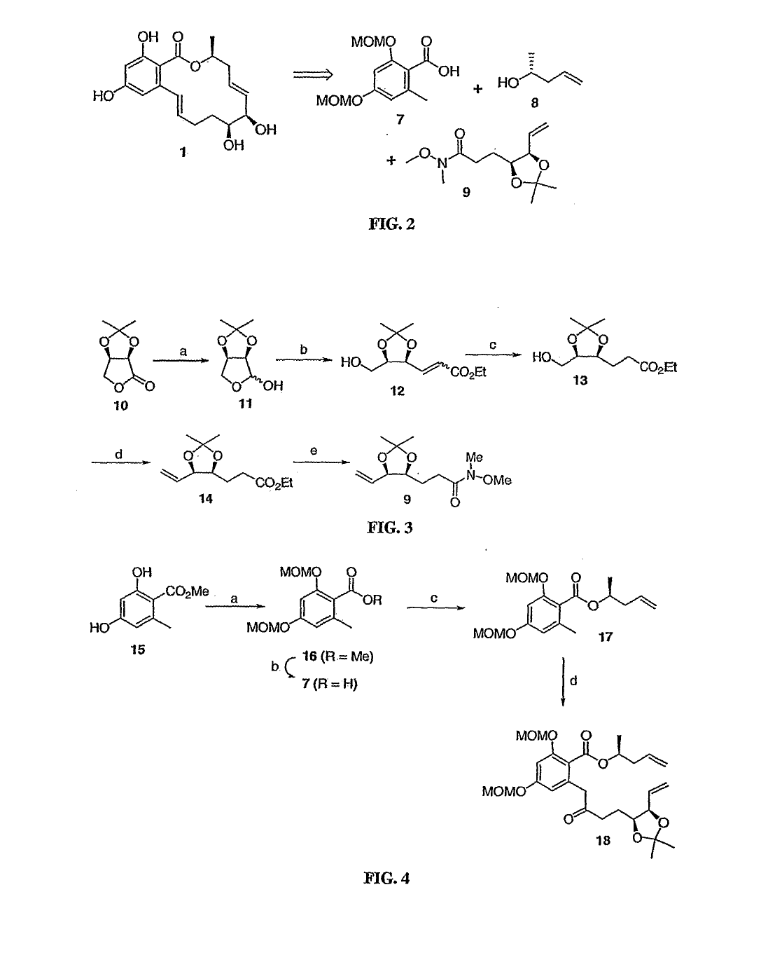

The above synthetic strategy is further demonstrated by the example shown for the synthesis of aigialomycin D (1) (FIG. 2) and selected compounds (2).

The synthesis of the amide (9) for acylation is shown in FIG. 3 (a. DIBAL-H, CH₂Cl₂, −78° C. then MeOH, 92%; b. Ph₃P═CHCO₂Et, PhCO₂H (0.2% mol), CH₂Cl₂, reflux, 73%; c. H₂(1 atm), 10% Pd/C (cat.), EtOH, 97%; d. (i) Dess Martin periodinane, DCM, rt; (ii) Ph₃P⁺CH₃Br⁻, ⁿBuLi, THF, −20° C. to rt, 52% over two steps; e. MeONHMe.HCl, ⁱPr₂MgCl, THF, −20° C. to rt, 80%.). The known acetonide (10) was reduced using DIBAL-H to provide the lactol (11) which was treated with ethyl (triphenylphosphoranylidene)acetate in the presence of a catalytic amount (0.2 mol %) of benzoic acid to afford the α,β-unsaturated ester (12) as a mixture of cis- and trans-isomer in 82% yield over two steps. Ester (12) was saturated under catalytic hydrogenation conditions to give the alcohol (13) which was oxidised using Dess Martin periodinane to give the corresponding aldehyde, which was further treated with methylenetriphenylphosphorane generated by treatment of methyltriphenyl-phosphonium bromide with n-butyl lithium to give the alkene ester (14) in 52% yield over three steps. The ester (14) was converted to the amide (9) in 80% yield by treatment with methoxymethylamine hydrochloride with two equivalents of isopropylmagnesiun bromide.

The synthesis macrocyclisation precursor (18) is shown in FIG. 4 (a. MOMCl, NaH, THF/DMF, rt, quant.; b. KOH, MeOH/H₂O 1/1, reflux then HOAc, pH4, 99%; c. DIAD, Ph₃P, 8, THF, rt, 82%; d. LDA, −78° C. THF then 9, 82%.). Methyl orcillinate was protected as its bis-MOM ether and further saponified to give the protected benzoic acid (7) which was coupled with the alcohol (4) under Mitsunobu conditions to give the ester (17). Alkylation at the position with the amide (9) was achieved by lithiation with LDA followed by reacting with (9) to give the ketone (18) in 82% yield.

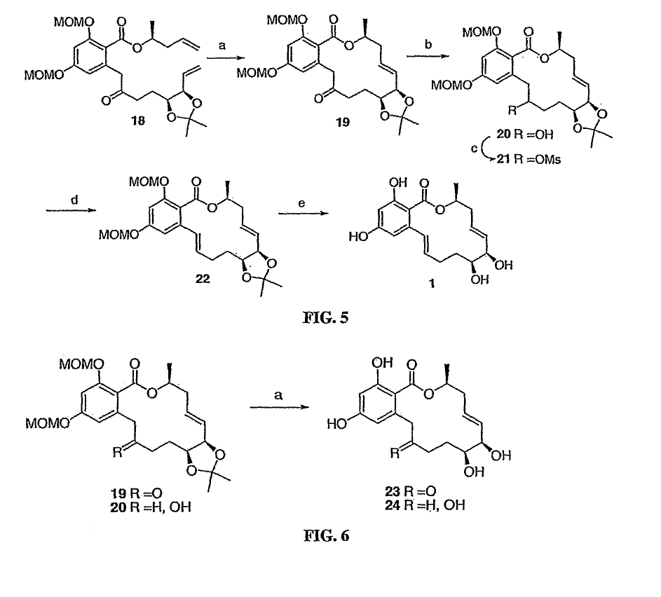

The synthesis of aigialomycin D (1) is shown in FIG. 5 (a. Grubb's II catalyst (20% mol), DCM, reflux, 2 days, 86%, E/Z=5.7:1 or. Grubb's II catalyst (10% mol), DCM, MWI, 100° C., 5 atm, 30 min, 98%, E-only; b. NaBH₄, MeOH/H2O (4:1), rt, quant.; c. (i) MsCl, Et₃N, DMAP (cat.), DCM; (ii) DBU, toluene, reflux, 74%; d. 1NHCl(aq.)/MeOH (1:1), rt, 2 days, 91%.).

The formation of the macrocycle from (18) was achieved by ring closing metathesis using Grubb's second generation catalyst. While reaction in reflux DCM afforded the cyclised product (19) [equivalent to (2) in (6′S, 7′S, 9′S)— configuration and wherein m=1, n=2, the dotted line is a single bond, R₁═R₃=OMOM, R₂═R₄═H, R₅ is absent, R₆═O, R₇═CMe₂, R₈ is a single bond, R₉═CH₃, H] in 86% yield with an E/Z ratio of 5.7:1 at the double bond, the cyclisation under microwave irradiation (MWI) conditions (100° C., 5 atm, 30 min) gave (19) in 98% yield with only the required E geometry.

Ketone (19) is a pivotal intermediate used for the synthesis of both aigialomycin D (1) and its selected analogues. For the synthesis of aigialomycin D (1), ketone (19) was reduced with sodium borohydride in methanol to give alcohol (20) [equivalent to (2) in (5′RS, 6′S, 7′S, 9′S)— configuration and wherein m=1, n=2, the dotted line is a single bond, R₁═R₃═OMOM, R₂═R₄═H, R₅ is absent, R₆H, OH, R₇═CMe₂, R₅ is a single bond, R₉═CH₃, H] in quantitative yield. Alcohol (20) was treated with methanesulfonyl chloride to form the corresponding mesylate (21) which was further treated with 1,8-Diazobicyclo[5.4.0]undec-7-ene (DBU) in toluene under reflux to give the diene (22) [equivalent to (2) in (6′S, 7′S, 9′S)— configuration and wherein m=1, n=2, R₁═R₃═OMOM, R₂R₄═H, R₅ and R₈ each is a single bond, R₆═H, R₇═CMe₂, R₉═CH₃, H] in 74% yield. Final global removal of all the protecting groups in (22) by treatment with hydrochloric acid in methanol afforded aigialomycin D (1) in 91% yield.

Synthesis of selected compounds (2) is shown in FIG. 6 (a. 1NHCl(aq.)/MeOH (1:1), rt, 2 days, 90% for (23); 86% for (24).).

From the key intermediate (19), a few selected compounds (2) were synthesised. Thus the protecting groups on ketone (19) was removed by treatment with hydrochloric acid in methanol to give compound (23) [equivalent to (2) in (6′S, 7′S, 9′S)— configuration and wherein m=1, n=2, the dotted line is a single bond, R₁═R₃═OH, R₂═R₄═H, R₅ is absent, R₆═O, R₇═H, H, R₈ is a single bond, R₉═CH₃, H] in 90% yield. In second example, the alcohol (20) was treated with hydrochloric acid in methanol to give compound (24) [equivalent to (2) in (5′RS, 6′S, 7′S, 9′S)— configuration and wherein m=1, n=2, the dotted line is a single bond, R₁═R₃—OH, R₂═R₄—H, R₅ is absent, R₆═H, OH, R₇═H, H, R₈ is a single bond, R₉═CH₃, H] in 86% yield.

### Synthesis of the Amide (9)

### (3aR,6aR)-Tetrahydro-2,2-dimethylfuro[3,4-d][1,3]dioxol-4-ol (11)

To a solution of D-erythronolactone acetonide (10) (10.0 g, 63 mmol) in dichloromethane (150 mL) was added via a dropping funnel a 1.0 M solution of diisobutylaluminum hydride in dichloromethane (100 mL, 100 mmol) at −78° C. under nitrogen. The resulting mixture was stirred for 3 h at −78° C., after which time methanol (32 mL) was added dropwise to destroy the remaining diisobutylaluminum hydride. Ethyl acetate (50 mL) and brine (30 mL) were added sequentially and the mixture was allowed to attend to room temperature. The mixture was acidified to pH 4 with 2M sulfuric acid and the jelly mixture was filtered though a pad of Celite®. The organic phase was separated and the aqueous phase was extracted with ethyl acetate (2×100 mL). The solid was allowed to dry overnight and filler extracted with ethyl acetate (3×400 mL) under ultrasonic irradiation. The combined extracts were dried (MgSO₄) and evaporated to give the title compound lactol 11 (9.32 g, 92%) as a viscous liquid, which was used for next step of reaction without further purification. Spectroscopic data were in agreement with those reported. R_{f} (EtOAc/n-hexane, 2/1) 0.44; IR (film) νₘₐₓ cm⁻¹ 3421, 2986, 2943, 2882, 1641, 1460, 1376, 1331, 1274, 1210, 1162, 1100, 1069, 1046, 986, 969, 926, 907, 874, 856, 817, 762, 666, 550, 516; ¹H-NMR (400 MHz, CDCl₃) δppm β/α=7.6/1. β: 5.41 (s, 1H, H₄), 4.83 (dd, 1H, ³J_{H6ax-H6a}=3.5 Hz, ³J_{H3a-H6a}=5.9 Hz, H₆ₐ), 4.56 (d, 1H, ³J_{H6a-H3a}=5.9 Hz, H₃ₐ), 4.06 (dd, 1H, ²J_{H6eq-H6ax}=10.3 Hz, ³J_{H6a-H6ax}3.5 Hz, H₆ₐₓ) 4.01 (d, 6H, ²J_{H6ax-H6eq}=10.3 Hz, H_{6eq}), 3.04 (br s, 1H, 4-OH), 1.46 (s, 3H, 2-CH₃), 1.31 (s, 3H, 2-CH₃). α: 4.98 (d, 1H, J_{H3a-H4}=7.0 Hz, H₄), 4.75 (dd, 1H, ³J_{H6ax-H6a}=3.7 Hz, ³J_{H3a-H6a}=6.2 Hz, H₆ₐ), 4.48 (m, 1H, H₃ₐ), 3.97 (d, 1H, ²J_{H6ax-H6eq}=11.0 Hz, H_{6eq}), 3.54 (dd, 1H, ²J_{H6eq-H6ax}=11.0 Hz, ³J_{H6a-H6ax}=3.7 Hz, H₆ₐₓ), 1.88 (br s, 1H, 4-OH), 1.54 (s, 3H, 2-CH₃), 1.37 (s, 3H, 2-CH₃).

### Ethyl 3-((4S,5R)-5-(hydroxymethyl)-2,2-dimethyl-1,3-dioxolan-4-yl)acrylate (12)

To a solution of (2R, 3R)-2,3-O-isopropylidene-D-erythrose (11) (i.e. lactol 11, above) (4.74 g, 30 mmol) in dichloromethane (150 mL) was added methyl (triphenylphosphoranylidene)acetate (12.5 g, 36 mmol) and benzoic acid (100 mg, 0.2% mol). The solution was refluxed for 17 h until TLC showed the lactol 11 had been consumed. The solvent was evaporated and the residue was triturated with 30% ether in hexane (4×100 mL). The combine extracts were evaporated and the residue was purified by chromatography on silica gel by gradient elution with ether-hexane to give the title compound (12) (4.96 g, 73%) as a mixture of E- and Z-isomers (E/Z1:2. Spectroscopic data were in agreement with those reported. R_{f} (EtOAc/n-hexane, 2/1) 0.53; [a]_{D}²⁴=+150.2 (c 0.418, EtOH); IR (film) νₘₐₓ cm⁻¹ 3441, 2986, 2938, 1714, 1645, 1456, 1416, 1381, 1303, 1259, 1217, 1194, 1164, 1047, 984, 923, 857, 684; ¹H-NMR (400 MHz, CDCl₃) δ ppm Z-isomer: 6.39 (dd, 1H, ³J_{H2-H3}=11.6 Hz, ³J_{H4′-H3}=7.0 Hz, H₃), 5.93 (dd, 1H, ³J_{H3-H2}=11.6 Hz, ³J_{H4′-H2}=1.7 Hz, H₂), 5.60 (ddd, 1H, ³J_{H5′-H4′}=7.2 Hz, ³J_{H3-H4′}=7.0 Hz, ³J_{H2-H4′}=1.7 Hz, H_{4′}), 4.58 (ddd, 1H, ³J_{H5′-H4′}=7.2 Hz, ³J_{CH2OH-H5′}=5.1 Hz, ³J_{CH2OH-H5′}=3.8 Hz, H_{5′}), 4.15 (q, 2H, J=7.1 Hz, 2H, OCH₂CH₃), 3.56 (dd, 1H, ²J=11.8 Hz, ³J_{CH2OH-H5′}=3.8 Hz, CH₂OH), 3.44 (dd, 1H, ²J=11.8 Hz, ³J_{CH2OH-H5′}=5.1 Hz, CH₂OH), 2.14 (br s, 1H, OH), 1.50 (s, 3H, 2′-CH₃), 1.38 (s, 3H, 2′-CH₃), 1.27 (t, 3H, J=7.1 Hz, OCH₂CH₃); E-isomer: 6.89 (dd, 1H, ³J_{H2-H3}=15.6 Hz, ³J_{H4′-H3}=5.6 Hz, H₃), 6.13 (dd, 1H, ³J_{H3-H2}=15.6 Hz, ³J_{H4′-H2}=1.6 Hz, H₂), 4.81 (ddd, 1H, ³J_{H5′-H4′}=7.1 Hz, ³J_{H3-H4′}=5.7 Hz, ³J_{H2-H4′}=1.6 Hz, H_{4′}), 4.37 (m, 1H, H_{5′}), 4.15 (q, 2H, J=7.1 Hz, 2H, OCH₂CH₃), 3.56 (dd, 1H, ²J=11.8 Hz, ³J_{CH2OH-H5′}=3.8 Hz, CH₂OH), 3.44 (dd, 1H, ²J=11.8 Hz, ³J_{CH2OH-H5′}=5.1 Hz, CH₂OH), 1.89 (br s, 1H, OH), 1.50 (s, 3H, 2′-CH₃), 1.38 (s, 3H, 2′-CH₃); 1.27 (t, 3H, J=7.1 Hz, OCH₂CH₃); ¹³C-NMR (100 MHz, CDCl₃) δ ppm Z-isomer: 166.0, 147.3, 121.1, 108.9, 78.8, 74.9, 61.5, 60.7, 27.4, 24.7, 14.2; E-isomer: 166.0, 142.1, 123.3, 109.7, 78.4, 62.0, 60.8, 27.8, 25.4, 14.4; MS-EI m/z calcd for C₁₁H₁₈O₅ [M-CH₃]⁺: 215.1, found: 215.1 (82%); HRMS-EI m/z calcd for C₁₁H₁₈O₅ [M-CH₃]⁺: 215.0919, found: 215.0916, Δ=−1.74 (ppm).

### Ethyl 3-[(4S,5R)-5-(hydroxymethyl)-2,2-dimethyl-1,3-dioxolan-4-yl]propanoate (13)

To a solution of the alkene ester (12) (7.2 g, 30 mmol) in ethanol (150 mL) was added 10% Pd on charcoal (1.5 g, 5 mol % Pd). The black mixture was evacuated and back-filled with hydrogen for four cycles and then stirred under H₂ (ca 1 atm) for 4.5 h until TLC showed the reaction had gone completion. The mixture was diluted with ethanol (100 mL) and filtered through a pad of Celite® and the residue washed with ethanol (2×30 mL). The filtrates were evaporated under reduced pressure to give the title compound (13) as a liquid (7.04 g, 97%). Spectroscopic data were in agreement with those reported. R_{f} (EtOAc/n-hexane, 2/1) 0.42; [a]_{D}²⁸=−21.4 (c 2.04, EtOH); IR (film) νₘₐₓ cm⁻¹ 3514, 2986, 2937, 1733, 1448, 1373, 1249, 1219, 1163, 1042, 926, 862, 801, 517; ¹H-NMR (400 MHz, CDCl₃) δ ppm 4.09-4.18 (m, 4H, OCH₂CH₃+H_{4′}+H_{5′}), 3.63 (d, 2H, J=4.5 Hz, CH₂OH), 2.51 (td, 1H, ²J=14.8 Hz, ³J_{H3-H2}=7.2 Hz, H₂), 2.38 (td, 1H, ²J=14.8 Hz, ³J_{H3-H2}=7.2 Hz, H₂), 2.14 (brs, 1H, OH), 1.80-1.83 (m, 1H, H₃), 1.43 (s, 3H, 2′-CH₃), 1.33 (s, 3H, 2′-CH₃), 1.23 (t, 3H, J=7.2 Hz, OCH₂CH₃); ¹³C-NMR (100 MHz, CDCl₃) δ ppm 173.2, 108.2, 77.7, 75.9, 61.5, 60.4, 31.1, 28.0, 25.4, 24.6, 14.1; MS-EI m/z calcd for C₁₁H₂₀O₅ [M-Me]⁺: 217.1 found: 217.1 (100%); HRMS-EI m/z calcd for C₁₁H₂₀O₅ [M-Me]⁺: 217.1076 found: 217.1063, Δ=−6.03 (ppm).

### Ethyl 3-[(4S,5R)-2,2-dimethyl-5-vinyl-1,3-dioxolan-4-yl]propanoate (14)

To a solution of the alcohol (230 mg, 1.00 mmol) (13) in CH₂Cl₂ (10 mL) was added Dess Martin periodinane (4 mL of 0.3 M solution in CH₂Cl₂, 1.2 mmol). The suspension was stirred at ambient temperature until (13) had been consumed (ca 4 h). Saturated NaHCO₃ (4 mL) and saturated Na₂S₂O₃ (4 mL) were added and the mixture was stirred for 10 min. The mixture was extracted with diethyl ether (3×10 mL), dried (MgSO₄) and evaporated to give crude aldehyde (160 mg) which was subjected to the next step Wittig reaction. R_{f} (EtOAc/n-hexane, 2/1) 0.50; IR (film) νₘₐₓ cm⁻¹ 3004, 2253, 1730, 1465, 1383, 1235, 1199, 1159, 1096, 907, 808, 762, 731, 650; ¹H-NMR (400 MHz, CDCl₃) δ ppm 9.67 (1H, d, J_{CHO-H5′}=3.1 Hz, 5′-CHO), 4.38 (ddd, 1H, J_{H3-H4′}=10.8 Hz, J_{H5′-H4′}=7.2 Hz, J_{H3-H4′}=3.8, H_{4′}), 4.30 (dd, 1H, J_{H5′-H4′}=7.2, J_{CHO-H5′}=3.1 Hz, H_{5′}), 4.14 (q, 2H, J=7.1 Hz, —OCH₂CH₃), 2.38-2.54 (m, 2H, H₂), 1.91-1.99 (m, 1H, H₃), 1.72-1.82 (m, 1H, H₂), 1.58 (s, 3H, 2′-CH₃), 1.40 (s, 3H, 2′-CH₃), 1.26 (t, 3H, J=7.1 Hz, —OCH₂CH₃); ¹³C-NMR (100 MHz, CDCl₃) δ ppm 201.9, 172.6, 110.7, 81.8, 77.4, 60.6, 30.9, 27.5, 25.3, 25.2, 14.1.

To a solution of the above aldehyde in THF (2 mL) cooled to −78° C. was added a solution of methylenetriphenylphosphorane generated by treatment of methyl triphenylphosphonium bromide with n-butyllithium (4 mL in THF, ca 0.5 M, 2.0 mmol) under argon. The temperature was allowed to rise to 0° C. and the yellow mixture was stirred at this temperature for 4 h. Acetone (1 mL) was added to the mixture and the mixture was allowed to stir at room temperature for 15 min. The solvent was evaporated and the residue was purified by column chromatograph to give the title compound (14) (117 mg, 52% over two steps) as colourless liquid; R_{f} (EtOAc/n-hexane, 1/1) 0.61; [a]_{D}²⁸=−20.6 (c 2.05, EtOH). IR (film) νₘₐₓ cm⁻¹ 2986, 2936, 1737, 1449, 1371, 1249, 1217, 1163, 1114, 1067, 931, 871; ¹H-NMR (400 MHz, CDCl₃) δ ppm 5.81 (ddd, Jₜᵣₐₙₛ=17.5 Hz, J_{cis}=10.3 Hz, J_{Hvinyl-H5′}=7.6 Hz, 5′-CH═CH₂), 5.33 (d, 1H, Jₜᵣₐₙₛ=17.5 Hz, 5′-CH═CH₂), 5.25 (d, 1H, J_{cis}=10.3 Hz, 5′-CH═CH₂), 4.53 (dd, 1H, J_{Hvinyl-H5′}=7.6 Hz, J_{H4′-H5′}=6.8 Hz, H_{5′}), 4.09-4.17 (m, 3H, OCH₂CH₃+H_{4′}), 2.33-2.51 (m, 2H, H₂), 1.71-1.77 (m, 2H, H₃), 1.47 (s, 3H, 2′-CH₃), 1.35 (s, 3H, 2′-CH₃), 1.25 (t, 3H, J=7.2 Hz, OCH₂CH₃); ¹³C-NMR (100 MHz, CDCl₃) δ ppm 173.3, 133.8, 118.6, 108.4, 79.5, 77.2, 60.4, 30.9, 28.1, 26.0, 25.6, 14.2; MS-EI m/z calcd for C₁₂H₂₀O₄ [M-Me]⁺ 213.1, found: 213.1 (27%) and 125.0 (100%), 98 (64%), 83 (33%); HRMS-EI m/z calcd for C₁₂H₂₀O₄ [M-CH₃]⁺ 213.1118, found: 213.1127, Δ=−3.96 (ppm).

### N-methoxy-N-methyl-3-((4S,5R)-2,2-dimethyl-5-vinyl-1,3-dioxolan-4-yl)propanamide (9)

To a slurry of Me(MeO)NH.HCl (0.61 g, 6.25 mmol) in THF (15 ml) was added a solution of ⁱPrMgCl in THF (6.25 ml of a 2.0M solution, 12.50 mmol) at −20° C. under argon. The mixture was stirred for 20 min to give a homogeneous solution to which a solution of the ester (14) (0.60 g, 2.63 mmol) in THF (5 nm) was added dropwise via a cannula. The reaction mixture was stirred at −20° C. for 1 hr before quenched with saturated NH₄Cl aqueous solution (10 mL). Upon warming to room temperature, the mixture was extracted with diethyl ether (2×30 mL). The combined extracts were washed with brine and dried (MgSO₄). Evaporation of the solvent followed by purification by flash column chromatography (SiO₂, EtOAc/n-hexane 1:1) provided the amide (9) (0.53 g, 83%) as a colorless oil; R_{f} (EtOAc/n-hexane, 3/1) 0.45; [a]_{D}^{24.5}=−27.3 (c 0.27, EtOH); IR (film) νₘₐₓ cm⁻¹ 2988, 2938, 2252, 1650, 1427, 1381, 1215, 1164, 995, 908, 733, 650; ¹H-NMR (400 MHz, CDCl₃) δ ppm 5.83 (ddd, Jₜᵣₐₙₛ=17.3 Hz, J_{cis}=10.3 Hz, J_{Hvinyl-H5′}=7.6 Hz, 5′-CH═CH₂), 5.33 (d, 1H, Jₜᵣₐₙₛ=17.3 Hz, 5′-CH═CH₂), 5.26 (d, 1H, J_{cis}=10.3 Hz, 5′-CH═CH₂), 4.55 (dd, 1H, J_{Hvinyl-H5′}=7.6 Hz, J_{H4′-H5′}=6.5 Hz, H_{5′}), 4.19 (ddd, 1H, J_{H3′-H4′}=9.5 Hz, J_{H5′-H4′}=6.5 Hz, J_{H3′-H4′}=4.6 Hz, H_{4′}), 3.69 (s, 3H, N—OCH₃), 3.18 (s, 3H, N—CH₃), 2.47-2.65 (m, 2H, H₂), 1.72-1.82 (m, 2H, H₃), 1.49 (s, 3H, 2′-CH₃), 1.37 (s, 3H, 2′-CH₃); ¹³C-NMR+DEPT 135 (100 MHz, CDCl₃) δ ppm 176.0, 134.0, 118.5, 108.3, 94.4, 79.6, 77.6, 61.2, 28.5, 28.2, 25.72, 25.68; MS-EI m/z calcd for C₁₂H₂₁NO₄ [M-CH₃]⁺: 228.1 found: 228.1 (40%); HRMS-EI m/z calcd for C₁₂H₂₁NO₄ [M-CH₃]⁺: 228.1236, found: 228.1228, Δ=−3.37 (ppm).

### Synthesis of the Macrocyclisation Precursor (18)

### 2,4-Bis(methoxymethoxy)-6-methylbenzoic acid (7)

To a solution of bis-MOM protected methyl ester (16), prepared according to a literature procedure, (1.35 g, 5.00 mmol) in MeOH (20 mL) was added KOH (1.40 g, 25.00 mmol) and H₂O (20 mL) at room temperature. The reaction mixture was heated at 90° C. for 2 days under argon. After cooling to room temperature, the mixture was acidified to pH 6 with acetic acid aqueous solution (50%; v/v) and extracted with EtOAc (3×100 mL). The combined organic phases were washed with H₂O (2×50 mL), dried over MgSO₄, filtered and evaporated under reduced pressure to afford (7) as a colorless oil (1.267 g, 99%), which was pure as judged by its ¹HNMR spectrum and was used immediately for next step of reaction. R_{f}(EtOAc/n-hexane, 1/1) 0.28; IR (film) νₘₐₓ cm⁻¹ 3020, 2963, 2832, 2402, 1698, 1606, 1448, 1398, 1317, 1294, 1215, 1150, 1047, 1029, 928, 756, 668; ¹H-NMR (400 MHz, CDCl₃) δ ppm 6.72 (s, 1H, H₅), 6.61 (s, 1H, H₃), 5.24 (s, 2H, OCH₂CH₃), 5.18 (s, 2H, OCH₂CH₃), 3.52 (s, 3H, OCH₂OCH₃), 3.48 (s, 3H OCH₂OCH₃), 2.47 (s, 3H, 6-CH₃); ¹³C-NMR (100 MHz, CDCl₃) δ ppm 174.6, 159.5, 156.6, 141.7, 116.0, 112.1, 101.4, 95.5, 94.2, 56.7, 56.3, 21.5; MS-EI m/z calcd for C₁₂H₁₆O₆ [M]⁺: 256.1, found: 256.0 (20%); HRMS-EI m/z calcd for C₁₂H₁₆O₆ [M]⁺: 256.0947, found: 256.0953., Δ=2.24 (ppm).

### (S)-Pent-4-en-2-yl 2,4-bis(methoxymethoxy)-6-methylbenzoate (17)

To a solution of (7) (1.02 g, 4.0 mmol), (R)-pent-4-en-2-ol (8) (0.516 g, 6.0 mmol) and triphenylphosphine (2.62 g, 10.0 mmol) in anhydrous THF (20 mL) was added dropwise a solution of DIAD (1.86 g, 9.2 mmol) in THF (5 mL) at room temperature. After stirring for 2 days under argon, the solvent was removed under reduced pressure and the crude product obtained was absorbed directly on silica gel (5 g), followed by column chromatography purification (EtOAc/n-hexane, 5-70% gradient) to afford (17) (1.06 g, 82%) as colorless oil: R_{f} (EtOAc/n-hexane, 1/9) 0.25; [a]_{D}^{23.5}=+11.7 (c 0.01, CHCl₃); IR (film) νₘₐₓ cm⁻¹ 2828, 2362, 1718, 1607, 1483, 1451, 1318, 1269, 1232, 1204, 1149, 1104, 1051, 1026, 994, 923, 838, 804, 756, 699, 664; ¹H-NMR (400 MHz, CDCl₃) δ ppm+COSY ¹H-¹H 6.66 (d, 1H, ⁴J=2.1 Hz, H₅), 6.54 (d, 1H, ⁴J=2.1 Hz, H₃), 5.84 (tdd, 1H, J=7.00, 10.2, 17.2 Hz, H_{4′}), 5.24 (qt, 1H, J=6.3, 12.6 Hz, H_{2′}), 5.08-5.14 (m, 6H, 2OCH₂OCH₃+2H_{5′}), 3.46 (s, 6H, 2OCH₂OCH₃), 2.33-2.50 (m, 2H, H_{3′}), 2.29 (s, 3H, 6-CH_{3),} 1.33 (d, 3H, J=6.3 Hz, H₁); ¹³C-NMR (100 MHz, CDCl₃) δ ppm+DEPT135 167.5, 158.5, 155.2, 137.6, 133.8, 119.1, 117.7, 110.6, 101.1, 94.6, 94.3, 70.9, 56.1, 56.0, 40.2, 19.7, 19.5; MS-EI m/z calcd for C₁₇H₂₄O₆ [M]⁺: 324.2, found: 324.2 (42%); HRMS-EI m/z calcd for C₁₇H₂₄O₆ [M]⁺: 324.1573, found: 324.1572, Δ=−0.38 (ppm); Anal. Calcd for C₁₇H₂₄O₆:C, 62.95; H, 7.46; Found: C, 62.85; H, 7.11.

### (S)-Pent-4-en-2-yl 2,4-bis(methoxymethoxy)-6-{4-[(4S,5R)-2,2-dimethyl-5-vinyl-1,3-dioxolan-4-yl]-2-oxobutyl}benzoate (18)

A solution of compound (17) (0.324 g, 1.00 mmol) in anhydrous THF (3 mL) was cooled to −78° C. and treated with freshly prepared LDA [diisopropylamine 0.303 g, 3.00 mmol), n-BuLi (1.56 mL 1.6M in hexane, 2.5 mmol), 5 mL THF], followed by immediate addition of compound (9) (0.291 g, 1.20 mmol) in THF (3 mL). The resulting mixture was stirred for 10 min at −78° C. and then quenched by addition of aqueous NH4Cl solution (3 mL). Upon warming to room temperature, the mixture was extracted with EtOAc (3×150 mL) and washed with H₂O (20 mL). The combined organic phases were dried over MgSO₄, filtered and evaporated to afford the crude product which was purified by flash chromatography (SiO₂, 5-100% EtOAc/n-hexane gradient) to provide (18) (0.417 g, 82%) as colorless oil; R_{f} (EtOAc/n-hexane, 1/1) 0.67; [a]_{D}^{24.3}=−6.5 (c 0.31, CHCl₃); IR (film) νₘₐₓ cm⁻¹ 2986, 2828, 1716, 1606, 1450, 1381, 1283, 1237, 1150, 1023, 924, 830, 803, 752, 688, 648; ¹H-NMR (400 MHz, CDCl₃) δ ppm+COSY ¹H-¹H 6.77 (d, 1H, ⁴J=2.1 Hz, H₅), 6.53 (d, 1H, ⁴J=2.1 Hz, H₃), 5.73-5.89 (m, 2H, H_{4″′}+5″-CH═CH₂), 5.07-5.32 (m, 9H, 2OCH₂OCH₃+5″-CH═CH₂+H_{5″′}+H_{2″″}), 4.49 (dd, 1H, J=6.7, 7.2 Hz, H_{5″}), 4.08-4.13 (m, 1H, H_{4″}), 3.74 (d, 1H, J=16.4 Hz, H_{1′}), 3.67 (d, 1H, J=16.4 Hz, H_{1′}), 3.47 (s, 3H, OCH₂OCH₃), 3.46 (s, 3H, OCH₂OCH₃), 2.51-2.68 (m, 2H, H_{3′}), 2.31-2.48 (m, 2H, H_{3″′}), 1.65-1.71 (m, 2H, H_{4′}), 1.45 (s, 3H, 2″-CH₃), 1.33 (s, 3H, 2″-CH₃), 1.30 (d, 3H, J=6.3 Hz, H_{1″′}); ¹³C-NMR (100 MHz, CDCl₃) δ ppm+DEPT135 206.4, 171.0, 170.0, 158.9, 156.2, 135.0, 134.0, 133.8, 118.4, 117.6, 111.3, 102.5, 94.7, 94.3, 79.5, 77.2, 71.1, 60.3, 56.2, 56.1, 47.9, 40.1, 38.3, 28.1, 25.5, 24.6, 21.0, 19.4, 14.1; MS-EI m/z calcd for C₂₇H₃₈O₉ [M]⁺: 506.3, found: 506.2 (24%); HRMS-EI m/z calcd for C₂₇H₃₈O₉. [M]⁺: 506.2516, found: 506.2503, Δ=−2.62 (ppm).

Synthesis of aigialomycin D (1)

### Ketone (19)

A 5 mM solution of (18) (0.152 g, 0.30 mmol) in anhydrous CH₂Cl₂ (60 mL) was treated with of Grubb's 2^{nd} generation catalyst (0.025 g, 10% mol) and stirred under microwave irradiation (100° C., 5 atm) for 30 min. After being cooled to room temperature, the solvent was removed under reduced pressure to afford the crude product which was purified by flash chromatography (SiO₂, 10-100% EtOAc/n-hexane gradient) to afford (19) as a light purple oil (0.140 g, 98%); R_{f} (EtOAc/n-hexane, 1/1) 0.50; [a]_{D}^{26.0}=+41.6 (c 0.339, CHCl₃); IR (film) νₘₐₓ cm⁻¹ 2982, 1718, 1605, 1585, 1270, 1218, 1148, 1102, 1042, 1018, 924, 772; ¹H-NMR (400 MHz, CDCl₃) δ ppm+COSY ¹H-¹H 6.75 (s, 1H, H₅), 6.53 (s, 1H, H₃), 5.77 (dd, 1H, J=6.8, 15.0 Hz, H_{8′}), 5.53 (dd, 1H, J=9.0, 15.0 Hz, H_{7′}), 5.33 (dd, 1H, J=6.3, 12.4 Hz, H_{10′}), 5.14 (s, 4H, OCH₂OCH₃), 4.47 (dd, J=6.4, 9.0 Hz, H_{6′}), 4.10-4.15 (m, 1H, H_{5′}), 3.82 (d, 1H, J=15.2 Hz, H_{1′}), 3.42-3.49 (m, 7H, 2OCH₂OCH₃+H_{1′}), 2.30-2.60 (m, 4H, H_{9′+}H_{3′}), 1.91-1.99 (m, 1H, H_{4′}), 1.68-1.77 (m, 1H, H_{4′}), 1.44 (s, 3H, 1″-CH₃), 1.39 (d, 3H, J=6.3 Hz, 10′-CH₃), 1.33 (s, 3H, 1″-CH₃); ¹³C-NMR (100 MHz, CDCl₃) δ ppm+DEPT135 205.9, 167.8, 158.9, 155.9, 133.8, 132.4, 129.0, 118.7, 110.7, 108.0, 102.1, 94.5, 94.2, 82.7, 76.3, 71.6, 56.2, 56.1, 47.2, 39.3, 37.6, 28.0, 25.2, 23.7, 20.8; MS-EI m/z calcd for C₂₅H₃₄O₉ [M]⁺: 478.2 found: 478.2 (47%); HRMS-EI m/z calcd for C₂₅H₃₄O₉ [M]⁺: 478.2203; found: 478.2203, Δ=−0.07 (ppm).

### Alcohol (20)

To a solution of (19) (0.065 g, 0.136 mmol) in 5 mL MeOH/H₂O (4/1) was added NaBH₄ (0.020 g, 0.54 mmol) portionwise at room temperature. The reaction mixture was stirred for 30 min and quenched with saturated solution of NH₄Cl (3 mL). The mixture was extracted with EtOAc (3×30 mL). The combined organic phases were dried over MgSO₄, filtered and evaporated under reduced pressure to afford (20) as colorless oil (0.067 g, 100%); R_{f} (EtOAc/n-hexane, 1/1) 0.36; [a]_{D}^{25.9}=−26.2 (c 0.052, CHCl₃); IR (film) νₘₐₓ cm⁻¹ 3489, 1981, 2934, 1718, 1604, 1583, 1449, 1399, 1379, 1269, 1216, 1147, 1040, 972, 923, 848, 754; ¹H-NMR (400 MHz, CDCl₃) δ ppm+COSY ¹H-¹H 6.70 (d, 1H, J=2.1 Hz, H₅), 6.66 (d, 1H, J=2.1 Hz, H₃), 5.72 (dd, 1H, J_{H8′-H9′}=6.8 Hz, J_{H8′-H7′}=15.4 Hz, H_{8′}), 5.59 (dd, 1H, J_{H7′-H6′}=9.2 Hz, J_{H8′-H7′}=15.4 Hz, H_{7′}), 5.35 (dd, 1H, J_{H10′-CH3}=6.2 Hz, J_{H10′-H9′}=12.5 Hz, H_{10′}), 5.14 (s, 4H, OCH₂OCH₃), 4.55 (dd, J_{H5′-H6′}=6.2 Hz, J_{H7′-H6′}=9.2 Hz, H_{6′}), 4.18-4.23 (m, 1H, H_{5′}), 3.89 (brs, 1H, H_{2′}), 3.46 (s, 6H, 2OCH₂OCH₃), 2.81 (dd, 1H, J_{H1′-H2′}=4.8 Hz, J_{H1′-H1′}=14.1 Hz, H_{1′}), 2.71 (dd, 1H, J_{H1′-H2′}=6.1 Hz, J_{H1′-H1′}=14.1 Hz, H_{1′}), 2.43-2.46 (m, 2H, H_{9′}), 1.67-1.80 (m, 4H, H_{3′}+H_{4′}), 1.46 (s, 3H, 1″-CH₃), 1.38 (d, 3H, J=6.2 Hz, 10′-CH₃), 1.35 (s, 3H, 1″-CH₃); ¹³C-NMR (100 MHz, CDCl₃) δ ppm+DEPT135 168.0, 158.6, 155.4, 138.0, 132.2, 130.2, 119.3, 110.6, 107.7, 101.4, 94.5, 94.3, 79.6, 77.2, 71.5, 70.0, 56.2, 56.1, 41.1, 39.6, 31.8, 28.1, 25.3, 24.7, 21.0; MS-EI m/z calcd for C₂₅H₃₆O₉ [M]⁺: 480.2 found: 480.2 (7%), 422.2 (32%), 325.1 (57%), 238.1 (100%); HRMS-EI m/z calcd for C₂₅H₃₆O₉ [M]⁺: 480.2359; found: 480.2358, Δ=−0.4 (ppm).

### Diene (22)

To a solution of (20) (190 mg, 0.396 mmol) in 5 mL of dry DCM was added Et₃N (400 mg, 3.96 mmol, 10 eq.) and DMAP (cat.) at room temperature under argon. To this mixture was added at 0° C. freshly distilled-MsCl (62 μL, 91 mg, 0.792 mmol, 2 eq.) dropwise. The mixture was stirred at ambient temperature for 5 h until the alcohol (20) was consumed. The solvent was removed under reduced pressure to give the crude mesylate (21) which was dissolved in toluene (20 mL) and DBU (0.59 mL, 601 mg, 3.96 mmol, 10 eq.) was subsequently added. The mixture was heated to reflux at 120° C. overnight. Toluene was removed under reduced pressure. The residue was extracted with EtOAc (3×30 mL). The combined organic phases were washed with water and dried over MgSO₄, filtered and evaporated under reduced pressure to afford diene (22) as colorless oil (135 mg, 74%); R_{f} (EtOAc/n-hexane, 1/1) 0.48; ¹H-NMR (400 MHz, CDCl₃) δ ppm 6.80 (d, 1H, J=2.0 Hz, H₅), 6.68 (d, 1H, J=2.0 Hz, H₃), 6.24 (d, 1H, J=15.4 Hz, H_{1′}), 6.14 (ddd, 1H, J_{H1′-H2′}=15.4 Hz, J_{H3′-H2′}=8.7 Hz, J_{H3′-H2′}=4.0 Hz, H_{2′}), 5.73 (ddd, 1H, J_{H8′-H9′}=3.6 Hz, J_{H8′-H9′}=9.1 Hz, J_{H8′-H7′}=15.5 Hz, H_{8′}), 5.59 (dd, 1H, J_{H7′-H6′}=9.6 Hz, J_{H8′-H7′}=15.5 Hz, H_{7′}), 5.30-5.37 (m, 1H, H_{10′}), 5.16 (s, 4H, OCH₂OCH₃), 4.56 (dd, J_{H5′-H6′}=5.4 Hz, J_{H7′-H6′}=9.6 Hz, H_{6′}), 4.16-4.21 (m, 1H, H_{5′}), 3.45 (s, 6H, 2OCH₂OCH₃), 2.45-2.55 (m, 2H), 2.29-2.32 (m, 1H), 2.07-2.11 (m, 1H), 1.80-1.85 (m, 1H), 1.49-1.55 (m, 1H), 1.46 (s, 3H, 1″-CH₃), 1.36 (d, 3H, J=6.2 Hz, 10′-CH₃), 1.35 (s, 3H, 1″-CH₃); ¹³C-NMR (100 MHz, CDCl₃) δ ppm 167.4, 158.9, 155.2, 136.8, 132.3, 131.9, 129.3, 128.5, 117.9, 108.3, 104.8, 102.6, 94.6, 94.3, 80.2, 77.3, 71.6, 56.2, 56.1, 39.5, 29.0, 28.7, 28.6, 25.9, 21.2.

### Aigialomycin D (1)

To a solution of the diene (22) (135 mg, 0.293 mmol) in MeOH (5 mL) was added HCl (1 N, 5 mL) and the mixture was stirred for 2 days at room temperature. The mixture was extracted with EtOAc (3×100 mL). The combined organic phases were washed with water until neutral, dried over MgSO₄, filtered and evaporated under reduced pressure to afford the crude aigialomycin D which was purified by flash chromatography (gradient from EtOAc/n-hexane 1/1 to EtOAc 100%) to afford aigialomycin D (1) as white solid (89 mg, 91%); R_{f} (EtOAc 100%) 0.52; [a]_{D}^{24.8}=−21.9 (c 0.3, MeOH); IR (film) νₘₐₓ cm⁻¹3391, 1646, 1456, 1312, 1259, 1167, 1110, 972; ¹H-NMR (400 MHz, CD₃COCD₃) δ ppm+COSY ¹ H-¹ H 11.67 (s, 1H, 2-OH), 9.23 (brs, 1H, 4-OH), 7.16 (d, 1H, J=15.9 Hz, H_{1′}), 6.53 (d, 1H, J=2.5 Hz, H₅), 6.28 (d, 1H, J=2.5 Hz, H₃), 6.10 (ddd, 1H, J_{H1′-H2′}=15.9 Hz, J_{H3′-H2′}=5.9 Hz, J_{H3′-H2′}=5.7 Hz, H_{2′}), 5.87 (dddd, 1H, J=15.6, 7.3, 7.3, 1.4 Hz, H_{8′}), 5.69 (dd, 1H, J_{H7′-H6′}=5.1 Hz, J_{H8′-H7′}=15.6 Hz, H_{7′}), 5.40-5.47 (m, 1H, H_{10′}), 4.36 (brd, J=4.7 Hz, H_{6′}), 3.78 (brs, 1H, 6′-OH), 3.63-3.66 (m, 1H, H_{5′}), 2.55 (ddd, J=14.6, 7.4, 3.3 Hz, H_{9′}), 2.32-2.36 (m, 2H, H_{3′}), 2.11-2.19 (m, 1H, H_{4′}), 1.58-1.61 (m, 1H, H_{4′}), 1.39 (d, 3H, J=6.4 Hz, 10′-CH₃); ¹³C-NMR (100 MHz, CDCl₃) δ ppm+DEPT135 172.3, 165.9, 163.3, 144.4, 135.8, 133.7, 130.8, 125.6, 108.0, 104.5, 102.7, 76.7, 73.4, 73.1, 38.1, 28.7, 28.1, 19.2.

### Synthesis of Macrolide (23-24)

### Macrolide (23)

To a solution of the ketone (19) (15 mg, 0.031 mmol) in MeOH (2 mL) was added HCl (1 N, 2 mL) and stirred for 2 days at room temperature. The mixture was extracted with EtOAc (3×50 mL). The combined organic phases were washed with water until neutral, dried over MgSO₄, filtered and evaporated under reduced pressure to afford the crude product which was purified by preparative TLC (EtOAc 100%) to afford macrolide (23) as colorless oil (9.8 mg, 90%); ¹H-NMR (400 MHz, MeOD) δ ppm+COSY ¹H-¹H 6.35 (d, 1H, J=2.5 Hz, H₅), 6.19 (d, 1H, J=2.5 Hz, H₃), 5.84 (ddd, 1H, J=15.3, 7.4, 7.2 Hz, H_{8′}), 5.69 (dd, 1H, J_{H7′-H6′}=5.9 Hz, J_{H8′-H7′}=15.3 Hz, H_{7′}), 5.32-5.36 (m, 1H, H_{10′}), 4.38-4.41 (m, 1H, H_{5′}), 3.96 (brd, 1H, J=5.2 Hz, H_{6′}), 3.93 (d, 1H, J=14.2 Hz, H_{1′}), 3.07 (d, 1H, J=14.2 Hz, H_{1′}), 2.41-2.63 (m, 2H, H_{9′}), 2.09 (dt, 2H, J=11.8, 2.9 Hz, H_{3′}), 1.59-1.71 (m, 1H, H_{4′}), 1.38-1.44 (m, 1H, H_{4′}), 1.41 (d, 3H, J=6.4 Hz, 10′-CH₃); ¹³C-NMR (100 MHz, MeOD) δ ppm+DEPT135 215.2, 170.0, 142.9, 134.3, 127.8, 114.8, 113.1, 112.7, 107.8, 102.4, 85.6, 76.0, 73.7, 40.3, 38.1, 33.7, 27.7, 20.2; MS-ESI m/Z calcd for Cl₁₈H₂₂O₇ [M-H]⁺: 349.1 found: 349.2 HRMS-EI m/z calcd for C₁₈H₂₂O₇ [M-H₂O]⁺: 332.1260 found: 332.1264, Δ=1.3 (ppm).

### Macrolide (24)

To a solution of the alcohol (20) (15 mg, 0.031 mmol) in MeOH (2 Ml) was added HCl (1 N, 2 mL) and stirred for 2 days at room temperature. The mixture was extracted with EtOAc (3×50 mL). The combined organic phases were washed with water until neutral, dried over MgSO₄, filtered and evaporated under reduced pressure to afford the crude product which was purified by preparative TLC (EtOAc 100%) to afford macrolide (24) as colorless oil (9.4 mg, 86%); ¹H-NMR (400 MHz, MeOD) δ ppm+COSY ¹H-¹H 6.23 (d, 1H, J=2.2 Hz, H₅), 6.20 (d, 1H, J=2.2 Hz, H₃), 5.74 (ddd, 1H, J=15.3, 6.9, 6.9 Hz, H_{8′}), 5.61 (dd, 1H, J_{H7′-H6′}=7.0 Hz, J_{H8′-H7′}=15.3 Hz, H_{7′}), 4.51-4.57 (m, 1H, H_{2′}), 3.91 (dd, 1H, J=6.3, 5.7 Hz, H_{6′}), 3.80 (tq, 1H, J=12.3, 6.1 Hz, H_{10′}), 3.48-3.52 (m, 1H, H_{5′}), 2.94 (dd, 1H, J=16.4, 3.8 Hz, H_{1′}), 2.85 (dd, 1H, J=16.4, 10.9 Hz, H_{1′}), 2.18-2.26 (m, 2H, H_{9′}), 1.79-2.02 (m, 3H, 2H_{3′}+H_{4′}), 1.43-1.53 (m, 1H, H_{4′}), 1.17 (d, 3H, J=6.1 Hz, 10′-CH₃); ¹³C-NMR (100 MHZ, MeOD) δ ppm+DEPT135 166.4, 143.5, 141.8, 133.1, 131.9, 130.9, 108.0, 102.2, 81.2, 77.1, 75.4, 68.4, 44.5, 43.2, 34.0, 32.4, 29.3, 23.1; MS-ESI m/z calcd for C₁₈H₂₄O₇ [M+Na]⁺: 375.1 found: 375.2; MS-ESI m/z calcd for C₁₈H₂₄O₇ [M-H]⁺: 351.1 found: 351.2.

1. A process for making compound 2: 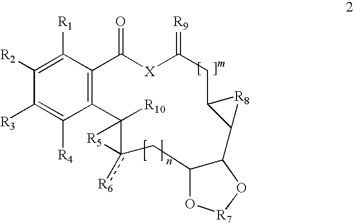
comprising the step of cyclizing diene 3A: 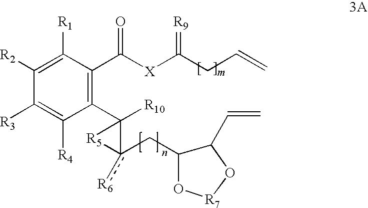
wherein:
m is an integer from 1 to 4; n is 0 or an integer from 1 to 4;
X is O, NRₐ wherein Rₐ is hydrogen, a protecting group, phenyl or an alkyl group of less than seven carbon atoms;
R₁ and R₃ are, independently, OR_{b}, OC(O)R_{b} or OCO₂R_{b}, wherein each R_{b} is independently hydrogen, a protecting group, optionally substituted phenyl or an alkyl group of less than seven carbon atoms;
R₂ and R₄ are, independently, hydrogen, halogen, nitro, cyano, SR_{c}, N(R_{c})₂ or NC(O)R_{c}, wherein each R_{c} is, independently, hydrogen, a protecting group, optionally substituted phenyl or an alkyl group of less than seven carbon atoms; [symbol]
represents either a single bond or a double bond whereby, if it is a double bond, R₅ is absent, and, if it is a single bond, R₅ is present;
whereby, if [symbol] is a single bond, R₅ is a single bond, O, CH₂, CF₂, NR_{d} or NC(O)R_{d} wherein R_{d} is hydrogen, phenyl or an alkyl group of less than seven carbon atoms and R₆ is a hydrogen, alkyl, aryl or heteroaryl;
whereby, if R₅ is absent, R₆ is O, CH₂, CF₂, (H, F), (F, F), N—ORₑ, (H, ORₑ), (OH, R_{f}) wherein Rₑ is hydrogen, alkyl sulfonyl, aryl sulfonyl or a protecting group, R_{f} is aryl, heteroaryl, alkyl or a perfluoroalkyl moiety of less than five carbon atoms;
R₇ is C═O, S═O, or a protecting group, or (H, H), or CRR′, wherein R and R′ are, independently, hydrogen, or an aryl, an alkyl or a cycloalkyl group;
R₈ is a single bond, O, CH₂, CF₂, NRₕ or NC(O)Rₕ wherein Rₕ is hydrogen, phenyl or an alkyl group of less than seven carbon atoms; and
R₉ is (H, R_{w}), where R_{w} is hydrogen, an alkyl group of less than 7 carbon atoms, an aryl group or a heteroaryl group;
R₁₀ is hydrogen or an alkyl or aryl group;
and wherein, where there is chirality at a position in compound 2 or diene 3A, the position may be in either R or S configuration or a mixture of both R and S configurations.

2. The process of claim 1 wherein diene 3A is diene 3:

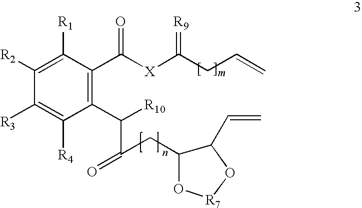

3. The process of claim 1 wherein the step of cyclizing is catalyzed by a Grubbs catalyst.

4. The process of claim 1 comprising the step of making diene 3A by coupling alkene I and amide II: 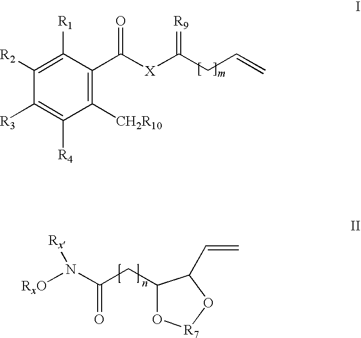
wherein Rₓ and R_{x′} are each, independently, an alkyl group of 1 to 6 carbon atoms.

5. The process of claim 4 wherein the step of coupling comprises lithiation of the benzylic methyl group of alkene I.

6. The process of claim 4 comprising the step of making alkene I by coupling benzoic acid 4 with alkene 5:

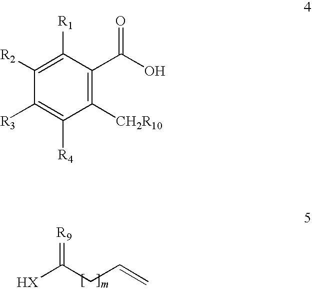

7. The process of claim 6 wherein the coupling of benzoic acid 4 with alkene 5 comprises a Mitsunobu reaction.

8. The process of any one of claims 1 additionally comprising elaboration of one or more functional groups of compound 2 so as to make aigialomycin D or a derivative thereof.

9. The process of claim 8 wherein the derivative is: 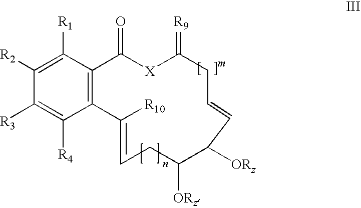
wherein:
R′₁, R′₂, R′₃, R′₄ and R′₉ are defined as for R₁, R₂, R₃, R₄ and R₉ respectively, and
R_{z}, and R_{z′} are, independently, hydrogen, a protecting group, phenyl or an alkyl group of less than seven carbon atoms or R_{z}, and R_{z′} together form a protecting group for a vicinal diol.

10. The process of claim 8 wherein the elaboration comprises deprotection of one or more functional groups.

11. The process of claim 8 wherein the elaboration comprises generation of a double bond in the non-aromatic ring.

12. A process for making a medicament for the treatment of cancer or malaria or a microbial infection comprising:
(A) making aigialomycin D or a derivative thereof by the process of any one of claims 1 to 11; and
(B) combining said aigialomycin D or derivative thereof with one or more pharmaceutically acceptable carrier, diluent and/or adjuvant.

13. A method of treating cancer or malaria or a microbial infection, said method comprising making a medicament by the process of claim 16 and administering a therapeutically effective dose of said medicament to a patient in need thereof.

14. Use of aigialomycin D or a derivative thereof according to claim 12 for the preparation of a medicament for the treatment of cancer or malaria or a microbial infection.

15. A method of treating cancer or malaria or a microbial infection, said method comprising administering to a patient in need thereof a therapeutically effective dose of aigialomycin D or a derivative thereof according to claim 12.

16. A process for making a medicament for the treatment of cancer or malaria or a microbial infection comprising:
(A) making aigialomycin D or a derivative thereof by the process of any one of claims 1 to 11; and
(B) combining said aigialomycin D or derivative thereof with one or more pharmaceutically acceptable carrier, diluent and/or adjuvant.

17. A method of treating cancer or malaria or a microbial infection, said method comprising making a medicament by the process of claim 16 and administering a therapeutically effective dose of said medicament to a patient in need thereof.
